SEQUENCE LISTING (8 sequences)

SEQ ID NO: 1 - protein, 1254 aa, Mycobacterium smegmatis
MGSSHHHHHHSSGLVPRGSHMMARIRLMSSSPSPFGQNEWLVEEMYRKFRDDPSSVDPSWHEFLVDYSPEPTTDSASNGRTTTAAPVTPPTPAPAPAPEPKAAPKPAAKTEAKPAKPAKSATPAKGDESQILRGAAAAVVKNMNASLEVPTATSVRAIPAKLMIDNRVVINNHLKRTRGGKISFTHLLGYAIVQAVKKFPNMNRHFAVVDGKPTAITPAHTNLGLAIDLQGKDGNRSLVVAAIKRCETMRFGQFIAAYEDIVRRARDGKLTAEDFSGVTISLTNPGTLGTVHSVPRLMQGQGAIIGAGAMEYPAEFQGASEERIADLGIGKLITLTSTYDHRIIQGAESGDFLRTIHQLLLDDDFFDEIFRELGIPYEPVRWRTDNPDSIEDKNARVIELIAAYRNRGHLMADIDPLRLDNTRFRSHPDLDVNSHGLTLWDLDREFKVDGFAGVQRKKLRDILSVLRDAYCRHVGVEYTHILEPEQQRWIQERVETKHDKPTVAEQKYILSKLNAAEAFETFLQTKYVGQKRFSLEGAETVIPMMDAVIDQCAEHGLDEVVIAMPHRGRLNVLANIVGKPYSQIFSEFEGNLNPSQAHGSGDVKYHLGATGTYIQMFGDNDIEVSLTANPSHLEAVDPVLEGLVRAKQDLLDTGEEGSDNRFSVVPLMLHGDAAFAGQGVVAETLNLALLRGYRTGGTIHIVVNNQIGFTTAPTDSRSSEYCTDVAKMIGAPIFHVNGDDPEACAWVARLAVDFRQAFKKDVVIDMLCYRRRGHNEGDDPSMTQPYMYDVIDTKRGSRKAYTEALIGRGDISMKEAEDALRDYQGQLERVFNEVRELEKHEIEPSESVEADQQIPSKLATAVDKAMLQRIGDAHLALPEGFTVHPRVRPVLEKRREMAYEGRIDWAFAELLALGSLIAEGKLVRLSGQDTQRGTFTQRHAVIVDRKTGEEFTPLQLLATNPDGTPTGGKFLVYNSALSEFAAVGFEYGYSVGNPDAMVLWEAQFGDFVNGAQSIIDEFISSGEAKWGQLSDVVLLLPHGHEGQGPDHTSGRIERFLQLWAEGSMTIAMPSTPANYFHLLRRHGKDGIQRPLIVFTPKSMLRNKAAVSDIRDFTESKFRSVLEEPMYTDGEGDRNKVTRLLLTSGKIYYELAARKAKENREDVAIVRIEQLAPLPRRRLAETLDRYPNVKEKFWVQEEPANQGAWPSFGLTLPEILPDHFTGLKRISRRAMSAPSSGSSKVHAVEQQEILDTAFG

SEQ ID NO: 2 - protein, 1064 aa, Scedosporium apiospermum
MGSSHHHHHHSSGLVPRGSHMMARIRLMLRNIACRAGGRLLSPSTSSPAWRSVSTRRAMAVASQKTRYTTSATSVPPDTSDNFLSGSAASYIDEMYMQWKEDPSSVHVSWQVYFRNMESGDMPISQAFIPPPNLVPSATGGVPSLTPGQGMGHSENSDLMKHLKVQLLCRAYQARGHHKANVDPLGIRNSSKGFGNVRPRELTLEFYNFTEKDLDAEFELGPGILPRFKTPERQKMTLREIISTLEHIYSGSFGVEFIHIPNRTRCDWLRERLEVPTPFKYSIDEKRRILDRLIWSSSFEAFLASKYPNDKRFGLEGCETLVPGMKALIDRSVDYGVKDIVIGMPHRGRLNVLSNVVRKPNESIFSEFAGTQSPDEGSGDVKYHLGMNFERPTPSGKRVQLSLVANPSHLEAEDPVVLGKTRAIQHYNNDESDHKTAMSVLLHGDAAFAAQGVVYECLGFHSLPAFSTGGTIHLVVNNQIGFTTDPRFARSTAYCTDIAKAIDAPVFHVNADDVEAVTFVCQLAADWRAEFQHDVVIDLICYRKHGHNETDQPSFTQPLMYKRIKDHEPQIDIYVDKLLREGTFTKEDIEEHKSWVWRMLQDSFNRSKDYQPTSKEWTTSAWNGFKSPKELATEILPHNPTAVDEQTLKHIGDVIGSYPEGFTVHNNLKRILKNRSKSVLDGAKNIDFPTAEALAFGSLVTEGNHVRVSGQDVERGTFSQRHAVFHDQETEDTYTPLQHLSKDQGKFVISNSSLSEFGALGFEYGYSLQSPNALVMWEAQFGDFANNAQCIIDQFVASGEVKWMQRTGLVMSLPHGYDGQGPEHSSGRLERYLQLSNEDPRIFPSEDKLARQHQDCNMQIVYMTEPSNLFHVLRRQMCRQFRKPLVIFFSKSLLRHPLARSSLDEFTGDSSFRWIIEDPEHKTGVIKSPEEIDRVILCTGQVYTALHKYRQDHQIDNVAFTRIEQLNPFPWQQLKENLDMYPNAKTIVWAQEEPLNAGAWSFTQPRIETLLNHTKYHDRKHVMYAGRNPSASVATGMKHVHTAEEAELLEMAFTVKQDKLKGEA

SEQ ID NO: 3 - protein, 1253 aa, Microbacterium maritypicum
MGSSHHHHHHSSGLVPRGSHMMARIRLMSNQVTGVNGDGGFGANSWLVEELYEQFKVNRDSVDKEWWPILEKYHSETASAAPAATPDAPAAAHPVTAPIPVIGSQPVARTTAKPAAAAPIPAQAPKPAKSESKDAPAPAEEDKVTPLRGLPKTLAANMDESLTVPTATSVRTVPAKLMIDNRIVINNHMARTRGGKVSFTHLIGWALIRTLDEFRSQNVFYAEVDGKPSVVAPAHVNLGIAIDLPKPDGTRALMVPSIKRADTLTFTEYLSAYEDLVTRARGNKLTAGDFQGTTVSLTNPGGIGTVHSVPRLMKGQGCIIGAGALEYPAEFQGASEKTLNELAIGKTITLTSTYDHRVIQGAGSGEFLKKVHELLIGQRGFYDDIFAALRIPYAPIRWNPDIAVDLAERVDKQSRVQELINSFRVRGHLMADIDPLEYVQRSHPDLEIESHGLTFWDLDREFVTGGFGGRRIAKLRDILGVLRDSYCRTLGIEYMHIQDPEQRRWFQEKVEVKYQKPGHDEQLRVLRKLNEAEAFETFLQTKFVGQKRFSLEGGESLIPLLDEILQGAATAGLEGAAIGMAHRGRLNVLTNIAGKTYGQVFREFEGTQTPGNQRGSGDVKYHLGTEGTFVADDDSELPVYLAANPSHLETVDGVLEGIVRAKQDRKPIGTFAWLPILVHGDAAFAGQGVVVETLQMSQLRGYRTGGTIHVVVNNQVGFTTTPNDGRTSIYSTDVAKTIQAPVFHVNGDDPEAVIHVAQLAFEYRERFHRDVVIDLVCYRRRGHNEGDDPSMTQPLMTDLIQAKRSVRRLYTESLVGRGDITEQEYDEAKADFQNRLEIAFAETHAAETGALPIAPEQAPVDDQVGAPEITGVPSEVIQMIGDAFVNKPEGFTVHPKIQQLLDKRLDMSRNGNIDWGFGELLAFGSLLVEGTPVRLAGQDARRGTFVQRHATLHDRANGQEWLPLSNLSDAQGRFFVYDSLLSEYAALGFEYGYSVEAPEALVLWEAQFGDFVNGAQSVIDEYISAAEQKWGQQSSVTLLLPHGYEGQGPDHSSARIERFLQMCAQDNMIVSRPSTPASYFHLLRRQAYARPRKPLIVFTPKAMLRLRGATSPVEAFTQGRFEPVIDDDRGLDRTAVKRVLVHSGKVHWDLRAELEKNPNPEVALVRLEQLYPTPIDELKAITDSYPNAELVWVQEEPENQGAWPFLALAFADVPGDRSFRPVSRPASASPATGSSKVHAAEQAALIRAAVTLG

SEQ ID NO: 4 - protein, 1296 aa, Arthrobacter enclensis
MGSSHHHHHHSSGLVPRGSHMMARIRLMPEQPSHRLPEEFGGNEWLVDELYEQYQQDKNAVDAKWWPLFESFDSGSSSSSNGHSAGAAANPPTTKLPVVNAAPAAPAPSPSAAPPAPAAPPAAAAPAAPAPVKKAPATEARDGGTKSSTGSGAQPIPAQLPKNVKAPTAPEEDVVSVLRGPAKAIATNMVTSLEVPTATSVRAIPAKLLIDNRVVINSNLARARGGKVSFTHLIGYAVIRALSQFPSMNVYYDEVDGKPVAVQPAHVNFGIAIDMPKPDGTRLLMVPNIKKAETLNFAEFWHTYEDLIKRARNGKLTAEDHQGTTVSLTNPGGIGTVHSVPRLSKGQAAIIGVGALDYPAEFQGASEKIIAQNAISKVLTLTSTYDHRVIQGAGSGEFLKLVHQLLLGAQNFYDEIFEALRIPYEPVRWSPDLQVDPADEINKVARIQQLIHSFRVRGHLMADTDPLEYVQRKHPDLDVLTYGLTLWDLDREWPTGGFGGKPMLKFRDILGVLRDAYCRTTGIEYMHIQEPAERKWFQDQLEHPYSKPSREEQLRIVSRLNAAEAFETFLQTKFVGQKRFSLEGGESLIPLLDAVISEAADDGLDEVAIGMAHRGRLNVLTNIAGKTYAQVFREFEGTQDPRSVQGSGDVKYHLGTEGTFTSENGNETKVYLAANPSHLEAVDSVLEGIVRAKQDRLDQGEAFPVLPIMVHGDAAFAGQGVVAETLNLSQLRGYRTGGTIHVVVNNQVGFTTAPSSSRSSTYSTDVAKMIQAPVFHVNGDDPEAVVRVGQLAYEFRQRFHKDVVIDMVCYRRRGHNEGDDPSMTQPMMYNLIEAKRSVRKLYTEALIGRGDITEEEAEQLLRDYQERLERVFAETHAAQTSPIPIVTADSAAVSDIERPTAQQADAGINAPASTAISADTLARIGHAHVEIPEGFTVHAKLKQLLEKREQMSREGGIDWGFGEIAAFGSLIMEGVPVRLAGQDSRRGTFVQRHAVFHDRANGNEWLPLGNLSEGQAKLWIYDSLLSEYAAMGFEYGYSVERPDALVLWEAQFGDFVNGAQTIIDEFISSAEQKWGQRSSLVLMLPHGYEGQGPDHSSARIERFLQLCAEQNMIVANPTTAASHFHLLRRQAYSRPRKPLIIFTPKQLLRLKAAASAVEDFTSGTFRPVIGEHEQLDANAVERVLLVSGRLYYDLLSTRQKTGDKTTAIIRVEQLYPLPHEEIAAELAKYPNAEVVWAQDEPANQGPWPFIGLNLPDLLDRRVRLVSRPASASTAAGSMKRHAAEQDVLLKQAFARK

SEQ ID NO: 5 - protein, 1274 aa, Kibdelosporangium phytohabitans
MGSSHHHHHHSSGLVPRGSHMMARIRLMSSSSPASQFGPNEWLVEEMYEQFLADPASVDPAWHDFFADYKPTQQRAQGVASNSATGQTTTVTKIEPPAPDTNGQAPAAKAAAPKATAPKAAQPKASAPSSAPPKPTTKPTKPATPAAKKPEPAKAAPQAEGDTSKPIRGAAAAIAKNMEQSLTVPTATSVRAVPAKLLFDNRVVINNRLRRTRGGKISFTHLIGYALVRALKDYPNMNRHYAAADGKPQLVTPEHVNLGLAIDLPGKDGARNLVVASIKGCEEMTFAQFWQAYEDLIRKARGGNLGTEDFAGTTISLTNPGTIGTNHSVPRLTAGQGAIIGVGAMEYPAQYQGTSEKALVEMGISKIMTLTSTYDHRIIQGAESGEFLRRIHQLLLGEDKFYDDVFTSLRIPYEPIRWVQDIPEGTLDKAARVIELIDAFRNRGHLMADTDPLNYRQRRHDDLDILSHGLTLWDLDREFAVGGFAGQERMKLRDVLSVLRDSYCRTVGIEYTHILDPAERRWIQDRVEIPHEKPDAAVQKYILSKLNAAEAFETFLQTKYVGQKRFSLEGGESVIPLLDTILDKAAEAELDEVVIGMPHRGRLNVLANIVGKPISQIFREFEGNLDPGQAHGSGDVKYHLGAEGKYFRMFGDGETKVSLTANPSHLETVDPVLEGIVRAKQDILDKGGEGFTVLPVALHGDAAFAGQGVVAETLNLALLRGYRTGGTVHVIINNQVGFTTAPEHSRSSQYATDVAKMIQAPVFHVNGDDPEACHWVARLAMDYRQAFNKDVVIDMLCYRRRGHNEGDDPSMTQPAMYDIIDTKRSVRKTYTEALIGRGDISVDEAEKALRDFSSQLEHVFNEVRELEKHPPVPSPSVEEEQQVPARVPTAVAREVIERIADAHINLPEGFTPHPRVKPVLERRAKMAREGDIDWAFGELLAFGSLNLEGKLVRLAGQDSRRGTFVQRHSVVVDRKTGQEYTPLQNLSEDQGRFMVYDSALSEYAAVGFEYGYSVANGDALVLWEAQFGDFVNGAQSVIDEFISSSEAKWGQLSDVVLLLPHGHEGQGPDHTSGRIERWLQLCAEGSMTVAIPSTPANYFHLLRRHALDGVTRPLIVFTPKSMLRNKAAVSPVEDFTNNKFMSVLDDPTQPDPQGVRKILFVSGKLYYELAAEQAKRGVTDTAIVRVEQLYPVPQKKLGLIFERYPNAHDVRWVQEEPANQGSWPFYGLFVREKFPERMGTMKRVSRRPMAAPSVGSSKVHEVEQRELIEKAFAD

SEQ ID NO: 6 - protein, 446 aa, Escherichia coli
MGSSHHHHHHSSGLVPRGSHMNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGEGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 7 - protein, 453 aa, Escherichia coli
MGSSHHHHHHSSGLVPRGSHMMARIRLMNSNKELMQRRSQAIPRGVGQIHPIFADRAENCRVWDVEGREYLDFAGGIAVLNTGHLHPKVVAAVEAQLKKLSHTCFQVLAYEPYLELCEIMNQKVPGDFAKKTLLVTTGSEAVENAVKIARAATKRSGTIAFSGAYHGRTHYTLALTGKVNPYSAGMGLMPGHVYRALYPCPLHGISEDDAIASIHRIFKNDAAPEDIAAIVIEPVQGSGGFYASSPAFMQRLRALCDEHGIMLIADEVQSGAGRTGTLFAMEQMGVAPDLTTFAKSIAGGFPLAGVTGRAEVMDAVAPGGLGGTYAGNPIACVAALEVLKVFEQENLLQKANDLGQKLKDGLLAIAEKHPEIGDVRGLGAMIAIELFEDGDHNKPDAKLTAEIVARARDKGLILLSCGPYYNVLRILVPLTIEDAQIRQGLEIISQCFDEAKQ

SEQ ID NO: 8 - protein, 368 aa, Rhodosporidium toruloides
MHSQKRVVVLGSGVIGLSSALILARKGYSVHILARDLPEDVSSQTFASPWAGANWTPFMTLTDGPRQAKWEESTFKKWVELVPTGHAMWLKGTRRFAQNEDGLLGHWYKDITPNYRPLPSSECPPGAIGVTYDTLSVHAPKYCQYLARELQKLGATFERRTVTSLEQAFDGADLVVNATGLGAKSIAGIDDQAAEPIRGQTVLVKSPCKRCTMDSSDPASPAYIIPRPGGEVICGGTYGVGDWDLSVNPETVQRILKHCLRLDPTISSDGTIEGIEVLRHNVGLRPARRGGPRVEAERIVLPLDRTKSPLSLGRGSARAAKEKEVTLVHAYGFSSAGYQQSWGAAEDVAQLVDEAFQRYHGAARESKL

(19) United States
(12) Patent Application Publication
(10) Pub. No.: US 2026/0146268 A1
(43) Pub. Date: May 28, 2026

(54) METHODS FOR IMPROVING YIELDS OF L-GLUFOSINATE

(71) Applicant: BASF SE, Ludwigshafen (DE)

(72) Inventors: Brian Michael Green, Lutherville, MD (US); Danielle Marie Estridge, Lutherville, MD (US)

(21) Appl. No.: 19/459,818

(22) Filed: Jan. 26, 2026

Related U.S. Application Data
(63) Continuation of application No. 17/272,803, now Pat. No. 12,559,777.
(60) Provisional application No. 62/727,322, filed on Sep. 5, 2018.

(51) Int. Cl. C12P 7/52

(52) U.S. Cl. CPC C12P 7/52; C12Y 206/01; C12Y 401/01071

(57) ABSTRACT

Compositions and methods for the production of L-glufosinate are provided. The method involves converting racemic glufosinate to the L-glufosinate enantiomer or converting PPO to L-glufosinate in an efficient manner. In particular, the method involves the specific amination of PPO to L-glufosinate, using L-glutamate, racemic glutamate, or another amine source as an amine donor. PPO can be obtained by the oxidative deamination of D-glufosinate to PPO (2-oxo-4-(hydroxy(methyl)phosphinoyl)butyric acid) or generated via chemical synthesis. PPO is then converted to L-glufosinate using a transaminase in the presence of an amine donor. When the amine donor donates an amine to PPO, L-glufosinate and a reaction by-product are formed. For example, when the amine donor is glutamate (including racemic glutamate or L-glutamate), L-glufosinate and the reaction by-product α-ketoglutarate (KG), also known as oxoglutarate, are produced. Because the PPO remaining represents a yield loss of L-glufosinate, it is desirable to minimize the amount of PPO remaining in the reaction mixture. Degradation, other chemical modification, extraction, sequestration, binding, or other methods to reduce the effective concentration of the by-product, i.e., the corresponding alpha ketoacid or ketone to the chosen amine donor will shift the reaction equilibrium toward L-glufosinate, thereby reducing the amount of PPO and increasing the yield of L-glufosinate.

Therefore, the methods described herein involve the conversion or elimination of the alpha ketoacid or ketone by-product to another product to shift the equilibrium towards L-glufosinate.

### CROSS-REFERENCE TO PRIORITY APPLICATION

This application claims is a continuation of U.S. patent application Ser. No. 17/272,803, which is a U.S. national phase of PCT/US2019/049467, filed Sep. 4, 2019, which claims the benefit of U.S. Provisional Application No. 62/727,322, filed Sep. 5, 2018; all of the aforementioned applications are hereby incorporated herein by reference in their entireties.

### INCORPORATION BY REFERENCE OF MATERIAL SUBMITTED ELECTRONICALLY

The Sequence Listing, which is a part of the present disclosure, is submitted concurrently with the specification as an .xml file. The name of the file containing the Sequence Listing is “M202647L_Seqlisting.xml”, which was created on Jan. 26, 2026 and is 15,309 bytes in size. The subject matter of the Sequence Listing is incorporated herein in its entirety by reference.

### FIELD

Described herein are methods for producing L-glufosinate in high yield.

### BACKGROUND

The herbicide glufosinate is a non-selective, foliarly-applied herbicide considered to be one of the safest herbicides from a toxicological or environmental standpoint. Current commercial chemical synthesis methods for glufosinate yield a racemic mixture of L- and D-glufosinate (Duke et al. 2010 Toxins 2:1943-1962). However, L-glufosinate (also known as phosphinothricin or (S)-2-amino-4-(hydroxy(methyl)phosphonoyl)butanoic acid) is much more potent than D-glufosinate (Ruhland et al. (2002) Environ. Biosafety Res. 1:29-37).

Therefore, methods are needed to produce only or primarily the active, L-glufosinate form. While a number of methods to generate pure L-glufosinate, or a mixture of D- and L-glufosinate enriched for L-glufosinate, have been described, cost effective methods to generate high yields of L-glufosinate from racemic glufosinate have not been available.

### SUMMARY

Described herein are new and cost-effective methods for the production of L-glufosinate in high yield. The methods involve converting racemic glufosinate to the L-glufosinate enantiomer. In particular, the methods involve the specific amination of PPO (2-oxo-4-(hydroxy(methyl)phosphinoyl)butyric acid) to L-glufosinate, using L-glutamate, racemic glutamate, or another amine source as an amine donor. PPO can be obtained by the oxidative deamination of D-glufosinate to PPO or generated via chemical synthesis. PPO is then converted to L-glufosinate using a transaminase in the presence of an amine donor. When the amine donor donates an amine to PPO, L-glufosinate and a reaction by-product are formed. For example, when the amine donor is glutamate (including racemic glutamate or L-glutamate), L-glufosinate and the reaction by-product α-ketoglutarate (KG), also known as oxoglutarate, are produced.

The transamination reaction is an equilibrium reaction, which means that under certain conditions, some PPO will remain when the reaction is at equilibrium. The PPO so remaining represents a yield loss of L-glufosinate, and thus it is desirable to minimize the amount of PPO remaining in the reaction mixture. Degradation, other chemical modification, extraction, sequestration, binding, or other methods to reduce the effective concentration of the by-product, i.e., the corresponding alpha ketoacid or ketone to the chosen amine donor, will shift the reaction equilibrium toward L-glufosinate, thereby reducing the amount of PPO and increasing the yield of L-glufosinate. Therefore, the methods described herein involve the conversion or elimination of the alpha ketoacid or ketone by-product to another product to shift the equilibrium towards L-glufosinate. The alpha ketoacid or ketone by-product can be converted by enzymatic or chemical means. The alpha ketoacid or ketone by-product can be reduced or eliminated by means such as ion exchange, size exclusion, or other resin. For example, when L-glutamate is used as the amine donor, the KG by-product can be converted to succinic semialdehyde (SSA) by the addition of a ketoglutarate decarboxylase (KGD) to the reaction. The reduction in the amount of KG available for conversion by the transaminase can lead to higher yields of L-glufosinate. If a product is generated by the reduction in amount or elimination of the by-product, it is called the conversion product. For example, if KG is the by-product and a KGD is used to reduce in amount or eliminate the KG, the conversion product is SSA.

Compositions described herein can comprise L-glufosinate and a conversion product. As discussed, where glutamate is used as the amine donor, KG is the by-product and SSA is the conversion product. In such compositions, L-glufosinate is present at a molar amount of 0.5 times to 1,000 times the molar amount of SSA or another conversion product.

Other compositions comprise L-glufosinate, PPO, and SSA. In such compositions, PPO is present at less than 10% of the amount of L-glufosinate and the conversion product is present at a molar amount up to 2 times the amount of L-glufosinate.

Also described herein is a method for selectively controlling weeds. In one embodiment, the method is useful for controlling weeds in a field containing a crop of planted seeds or crops that are optionally resistant to glufosinate. The method comprises applying an effective amount of a composition comprising L-glufosinate at an enantiomeric excess of greater than 90% over D-glufosinate to the field. Such methods of use include selectively controlling weeds in a field, controlling weeds in non-field areas, defoliating plants or crops, and/or desiccating crops before harvest, comprising applying an effective amount of a composition comprising L-glufosinate at an enantiomeric excess of greater than 90% over D-glufosinate and more than 0.01% but less than 10% PPO and less than about 10% of the ketone byproduct. In one embodiment, the composition comprises greater than 90% L-glufosinate, less than 10% PPO, and less than 10% of the ketone byproduct conversion product relative to the amount of L-glufosinate. In some embodiments, the ketone byproduct conversion product is SSA.

The details of one or more embodiments are set forth in the drawings and the description below. Other features, objects, and advantages will be apparent from the description and drawings, and from the claims.

### BRIEF DESCRIPTION OF THE DRAWINGS

FIG. 1 is a schematic of an exemplary conversion of racemic glufosinate to L-glufosinate using L-glutamate as an amine donor and a ketoglutarate decarboxylase (KGD) as the method to reduce the amount of KG.

FIG. 2 is a schematic of the reaction in FIG. 1 if the chosen transaminase (TA) is capable of converting L-glutamate and SSA to KG and gamma-aminobutyric acid (GABA); if the TA is capable of this conversion at a high rate relative to the conversion of PPO and L-glutamate to KG and L-glufosinate, the method will not yield high levels of L-glufosinate.

FIGS. 3A-C are graphs showing concentrations of L-glufosinate (black circles, black bold line), D-glufosinate (light grey circles, light grey solid line), PPO (dark grey diamonds, dark grey solid line), L-glutamate (black triangles, black dashed line), and KG (dark grey squares, dark grey dashed line) during a deracemization of racemic glufosinate utilizing a DAAO enzyme and a TA enzyme at two starting concentrations of L-glutamate without a KGD enzyme at 300 mM L-glutamate (FIG. 3A) and with a KGD enzyme at 300 mM L-glutamate (FIG. 3B) or 150 mM L-glutamate (FIG. 3C).

### DETAILED DESCRIPTION

Compositions and methods for the production of L-glufosinate (also known as phosphinothricin or (S)-2-amino-4-(hydroxy(methyl)phosphonoyl)butanoic acid) are provided. In particular, the methods described herein increase the yield of L-glufosinate or simplify the isolation of L-glufosinate from the reaction mass. When the starting material for the production of substantially purified L-glufosinate is racemic glufosinate, the methods comprise a three-step process, which may occur in a single vessel and nearly simultaneously, in multiple vessels and sequentially, or in multiple vessels and nearly simultaneously.

U.S. Pat. No. 9,834,802, herein incorporated by reference, describes a two-step method for the production of L-glufosinate comprising reacting D-glufosinate with a D-amino acid oxidase (DAAO) enzyme to form PPO (2-oxo-4-(hydroxy(methyl)phosphinoyl)butyric acid); followed by aminating the PPO to L-glufosinate by a transaminase (TA) enzyme, using an amine group from one or more amine donors. The reaction of PPO with a transaminase results in the production of L-glufosinate and a by-product, the corresponding alpha ketoacid or ketone, depending upon the chosen amine donor. Suitable amine donors include L-aspartate or racemic aspartate, L-glutamate or racemic glutamate, L-alanine or racemic alanine, L-1-phenylethylamine or racemic 1-phenylethylamine, L-phenylalanine or racemic phenylalanine, phenylethylamine, L-glycine or racemic glycine, L-lysine or racemic lysine, L-valine or racemic valine, L-serine or racemic serine, L-glutamine or racemic glutamine, isopropylamine, sec-butylamine, ethanolamine, 2-aminobutyric acid, and diaminopropionic acid.

The present method adds a third step to the method set forth in U.S. Pat. No. 9,834,802, described above, that involves reducing the effective levels of the alpha ketoacid by-product generated during the second step of amination of PPO to L-glufosinate. The by-product of the second step can be removed by enzymatic or chemical conversion, by physical removal such as ion exchange, size exclusion, and the like. Without being bound by theory, it is thought that by reducing the effective levels of the by-product of the second step, the second reaction can continue at a higher rate and/or reach equilibrium concentrations that result in a higher yield of L-glufosinate. By combining these reactions, the proportion of L-glufosinate can be substantially increased in a racemic glufosinate mixture. Thus, provided herein are methods to obtain a composition substantially comprising L-glufosinate. For purposes of the methods and compositions described herein, a composition comprising substantially L-glufosinate contains less than 10% PPO, less than 5% PPO, less than 2% PPO, less than 1% PPO, contains at least 0.01% PPO, or contains no detectable PPO, relative to the amount of L-glufosinate. Additionally, the composition comprises an amount of the α-ketoacid or ketone by-product (for example, KG). For purposes of the methods and compositions described herein, a composition comprising substantially L-glufosinate contains less than 50% by-product, less than 25% by-product, less than 10% by-product, less than 5% by-product, less than 2% by-product, less than 1% by-product, or contains no detectable by-product, relative to the amount of L-glufosinate. Reduction in the amount of the by-product results in increasing levels of the conversion product. Therefore, the composition may comprise at least 5% conversion product, at least 10% conversion product, at least 25% conversion product, at least 50% conversion product, at least 75% conversion product, at least 100% conversion, or at least 200% conversion product relative to the amount of L-glufosinate. Since L-glufosinate is more potent than D-glufosinate, smaller amounts of the composition are needed to be effective as an herbicide.

The methods described herein provide an efficient method for the conversion of PPO to L-glufosinate, particularly when a transaminase is used in the reaction. As discussed, the transaminase produces L-glufosinate and an alpha ketoacid or ketone by-product. The methods described herein provide for the reduction of the alpha ketoacid or ketone by-product to drive the reaction towards L-glufosinate, reducing the amount of PPO. A number of amine donors can be used in the reaction. If glutamate or L-glutamate is the amine donor, the by-product would be the alpha ketoacid KG. If isopropylamine is the amine donor, the by-product would be the ketone acetone. If alanine is the amine donor, the by-product would be the alpha ketoacid pyruvate. If sec-butylamine is the amine donor, the by-product would be the ketone 2-butanone. If phenylethylamine is the amine donor, the by-product would be the ketone acetophenone. If lysine is the amine donor, the by-product would be the alpha ketoacid hexanoic acid or the semialdehyde 2-aminoadipate-6-semialdehyde. If aspartate is the amine donor, the by-product would be the alpha ketoacid oxaloacetate. If glycine is the amine donor, the by-product would be the alpha ketoacid glyoxylate. If valine is the amine donor, the by-product would be the alpha ketoacid alpha ketoisovaleric acid. If serine is the amine donor, the by-product would be the alpha ketoacid 3-hydroxypyruvate. If glutamine is the amine donor, the by-product would be the alpha ketoacid 2-oxoglutaramate. If ethanolamine is the amine donor, the by-product would be the aldehyde glycolaldehyde. If 2-aminobutyric acid is the amine donor, the by-product would be the alpha ketoacid 2-oxobutanoic acid. If diaminoproprionic acid is the amine donor, the by-product would be the alpha ketoacid 3-amino-2-oxopropanoic acid or the semialdehyde 3-oxoalanine.

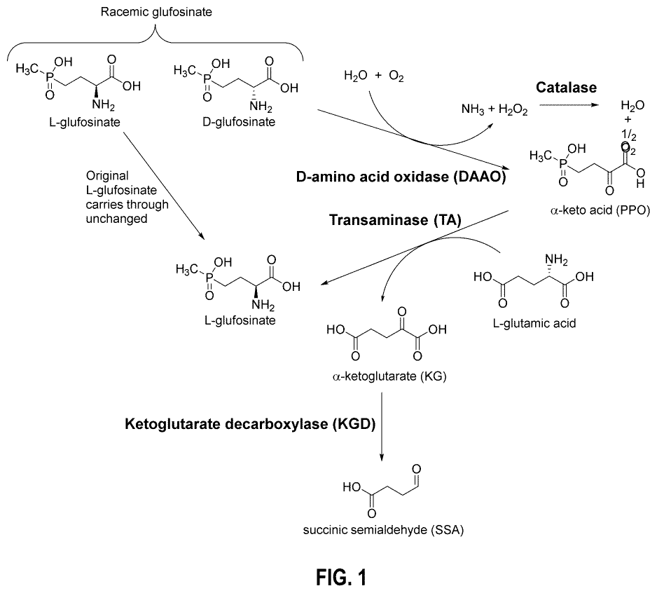

In one embodiment, the first step is catalyzed by a D-amino acid oxidase (DAAO) enzyme and the second step is catalyzed by a transaminase (TA) enzyme and glutamate is utilized by the TA enzyme as the amine donor. The by-product produced is α-ketoglutarate (KG). In this embodiment, a ketoglutarate decarboxylase (KGD) enzyme can be used, for example, (EC 4.1.1.71). Without intending to be limiting, SEQ ID NO:1 through SEQ ID NO:5 are example KGD enzymes. The ketoglutarate decarboxylase converts KG to carbon dioxide and succinic semialdehyde (SSA). FIG. 1 contains a schematic of this exemplary reaction. While the methods can be used to produce a substantially purified L-glufosinate in a batch reaction, it is recognized that a continuous process can be used.

One embodiment, described herein, is a composition comprising a mixture of L-glufosinate and SSA. Initially, the amount of L-glufosinate will be much higher than the amount of SSA. As the reaction proceeds, the amount of SSA increases, and could reach as high as the amount of L-glufosinate, or even higher. Compositions of L-glufosinate may comprise L-glufosinate and SSA, with the molar amount of L-glufosinate being 0.5 times to 1,000 times the amount of SSA. These compositions can optionally occur as dried powders or dissolved in aqueous or non-aqueous carriers and additional chemical species can optionally be present. Optionally, the composition is prepared and used in an ex vivo environment.

It is also recognized that the L-glufosinate can be further isolated and used in formulations as an herbicide. In many of the reactions to generate L-glufosinate that are known in the art, the post-reaction mixture contains a number of additional components, often times at significant levels relative to L-glufosinate. The complexity of these mixtures can make it difficult and or costly to isolate the L-glufosinate. In the case of the reactions described in U.S. Pat. No. 9,834,802, the post-reaction mixtures typically include L-glufosinate, KG, PPO, and glutamic acid. Isolation of L-glufosinate in the post-reaction mixture typically requires multiple operations because the chemical structures and chemical properties of these components are very similar. L-glutamic acid presents the main challenge because it is present in a high concentration relative to L-glufosinate and is structurally similar to L-glufosinate. In the International PCT Patent Application PCT/US2018/042503, “Methods for the Purification of L-Glufosinate,” incorporated herein by reference in its entirety, improved methods of isolation are described, but even simpler isolation would be preferred. It is recognized that the reactions embodied here could have lower levels of KG, PPO, and glutamic acid, and higher levels of L-glufosinate, than the reactions described in U.S. Pat. No. 9,834,802, and that this could simplify the isolation of L-glufosinate.

### I. Methods of Synthesis

Methods for the conversion of D-glufosinate to L-glufosinate at high yield in an ex vivo environment are provided. The methods described herein provide a means for converting a low-cost feedstock of a racemic mixture of D- and L-glufosinate into a more valuable product that has been enriched for L-glufosinate.

FIG. 1 shows an exemplary schematic of one embodiment described herein. In this example, a DAAO enzyme, a TA enzyme, and a ketoglutarate decarboxylase (KGD) enzyme are combined, optionally with a catalase enzyme, to catalyze several reactions that lead to an enrichment of L-glufosinate from racemic glufosinate. These reactions could occur in one, or more than one, reaction vessel, including in batch or flow reaction modes. These enzymes could be added at the same time (including by co-immobilization to a solid support) or at different times as desired.

If the reaction shown in FIG. 1 does not contain the KGD enzyme, approximately 3-4 times as many moles of glutamate as moles of racemic glufosinate are required at the start of the reaction to bias the equilibrium sufficiently toward L-glufosinate that yield losses to PPO are less than 10% (see U.S. Pat. No. 9,834,802). When the effective levels of KG available to the TA are reduced, as in the methods described herein, the equilibrium is more biased toward L-glufosinate. In fact, complete conversion of PPO to L-glufosinate could be obtained with an equivalent number of moles of glutamate as moles of PPO (which is half the number of moles of racemic glufosinate before the DAAO enzyme catalyzes the oxidative deamination of the D-glufosinate) if the KGD enzyme is able to completely, and solely, catalyze the decarboxylation of KG to SSA and if SSA is not a substrate for the TA enzyme. This reaction could completely consume the input glutamate, the by-product KG, and convert all produced PPO to L-glufosinate. The final reaction mass could include SSA at 0.5 times the molar amount of L-glufosinate and no other major organic non-enzymatic reaction components. This simplified reaction mass would clearly provide advantages for purification and isolation of L-glufosinate.

Ketoglutarate decarboxylase (KGD) enzymes (EC 4.1.1.71) catalyze the conversion of KG to carbon dioxide and SSA. KGD enzymes are well known in the art and can be found in a wide range of organisms, including M. smegmatis, S. apiospermum, M. maritypicum, A. enclensis, and K. phytohabitans. Without intending to be limiting, SEQ ID NO:1 through SEQ ID NO:5 are example KGD enzymes. In a preferred embodiment, the KGD enzyme is based on the KGD enzyme from A. enclensis. Since KG is an α-ketoacid and PPO is an α-ketoacid and the decarboxylation occurs from the carboxylic acid at the same end of these relatively similar molecules, it is preferred to utilize a KGD enzyme that rapidly decarboxylases KG but does not efficiently catalyze the decarboxylation of PPO. It may be useful to use various enzyme improvement techniques well known in the art to improve the efficiency of catalysis of the decarboxylation of KG, reduce the efficiency of catalysis of the decarboxylation of PPO, or both. KGD enzymes are examples of a broader class of carboxy-lyase enzymes, as represented by all of the E.C. numbers starting with 4.1.1, and any of those carboxy-lyase enzymes could represent enzymes that could reduce the amount of the by-product and provide the advantages described herein.

In some embodiments, the SSA is sequestered from the reaction mass. This could be preferred to avoid unwanted reactivity of SSA with other reaction components, to enhance the rate of conversion of KG to SSA, or to initiate the purification and isolation of L-glufosinate. SSA can be removed by methods known in the art such as binding to a resin (such as an amine resin, ion exchange resin, size exclusion resin, or other resin), conversion to an oxime (such as with a hydroxylamine derivative) with optional precipitation, reduction (such as with NaBH4), polymerization, or oxidization. SSA that has been produced by a KGD enzyme can be further oxidized to succinic acid, an important chemical intermediate.

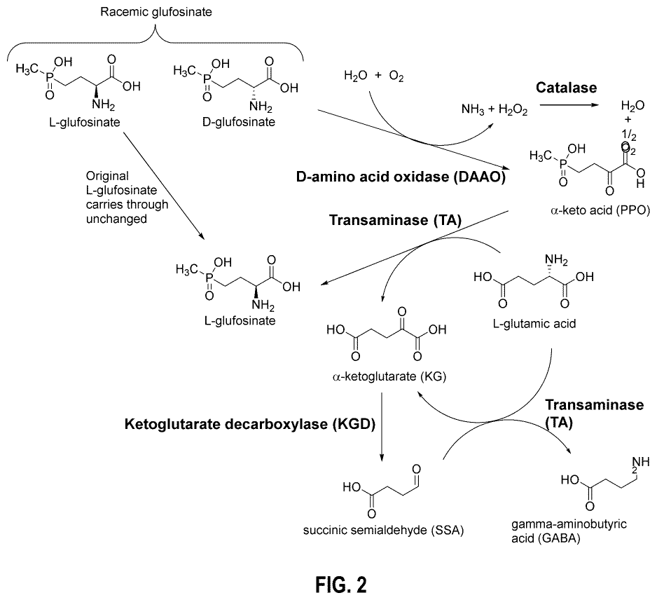

Decarboxylation of KG to SSA is intended to reduce the effective concentration of KG available to the TA and thereby shift the reaction equilibrium toward L-glufosinate and KG from PPO and glutamate. It is possible, however, that SSA could be utilized by enzymes in the reaction mass other that the KGD enzyme. In fact, the reaction natively catalyzed by the E. coli gabT TA enzyme commonly utilized in U.S. Pat. No. 9,834,802 is to convert SSA and L-glutamate to KG and gamma-aminobutyric acid (GABA). FIG. 2 shows a schematic of this reaction as part of the full L-glufosinate deracemization reaction. Without being bound be theory, it is thought that such a reaction, when combined with the reaction catalyzed by a KGD enzyme, could eventually lead to depletion of glutamate and accumulation of GABA. This could lead to increased levels of PPO and decreased levels of L-glufosinate, which is counter to the objectives described herein.

In a preferred embodiment, TA enzymes or enzyme mutants or variants are used that do not efficiently utilize the conversion product that is generated by the reduction in amount or elimination of the by-product produced when L-glufosinate is aminated. Mutant variants of gabT have been described that demonstrate varying affinities for substrates including SSA (Liu et al., Biochemistry 2005, 44, 2982-2992). In a preferred embodiment, TA enzymes that do not efficiently catalyze the conversion of SSA and glutamate to KG and GABA, but do efficiently catalyze the conversion of PPO and glutamate to KG and L-glufosinate, are used. In a more preferred embodiment, an E211S mutation in the E. coli gabT TA enzyme (SEQ ID NO:6 is the wild type TA enzyme with leader sequence, SEQ ID NO:7 is the E211S mutant TA enzyme with leader sequence) is used. Sequence homology can be used to identify enzymes or mutations that would be expected to confer the desired substrate specificity. For example, the top 100 sequences found via a blastp search of the NCBI non-redundant sequence database on Aug. 31, 2018 with the wild type E. coli gabT TA enzyme sequence, excluding all hits from Enterobacteraceae, yielded sequences from 6 genus. Despite sequence identity as low at 76% compared to the E. coli gabT TA enzymes sequence among these hits (GenBank accession number CUJ92856.1 is 100%, KKF68526.1 is 91%, WP_061553513.1 is 89%, WP_025801403.1 is 89%, WP_074941369.1 is 76%, and PRD29013.1 is 76%) all contain a glutamic acid at the equivalent of position E211 in the E. coli sequence, when aligned using Cobalt, and would therefore be predicted to demonstrate the desired activity if the glutamic acid were converted to serine.

Structural homology can also be used to identify amino acids in the active site of the TA enzyme that serve a similar coordination role as E211 does in the E. coli gabT enzyme and would therefore be predicted to confer a similar shift in substrate specificity when mutated. Phylogenetically distinct collections of TA enzymes can be screened to identify those that efficiently catalyze the desired reaction (conversion of PPO to L-glufosinate) and less efficiently catalyze the undesired reaction (accepting the conversion product as a substrate, for example, SSA). Therefore, TA enzymes having a serine at the equivalent position of E211 when aligned with the E. coli gab T TA (SEQ ID NO:6) may be used in the methods described herein. It may also be useful to use various enzyme improvement techniques well known in the art to further improve the desired activity, reduce the undesirable activity, or both.

Additional methods to reduce the levels of KG include non-enzymatic oxidation by various means, such as hydrogen peroxide, reduction, either enzymatic or non-enzymatic, and degradation catalyzed by an ethylene synthetase. In CN107119084A, a method is described to reduce KG using an ethylene synthase, which catalyzes the conversion of 3 KG, 3 O₂, and 1 arginine to 2 ethylene, 1 succinate, 1 guanidine, 1 pyrroline-5-carboxylate, 7 carbon dioxide, and 3 H₂O. While the use of an ethylene synthetase can reduce the effective amount of KG, it requires the addition of several co-substrates and produces a number of products that must be removed from the reaction mass.

A higher yield of L-glufosinate can also be achieved by the enzymatic decarboxylation of α-ketoglutarate to succinate (succinic acid) by alpha-ketoglutarate dependent dioxygenases. These iron-containing enzymes catalyze a wide range of oxygenation reactions using oxygen and α-ketoglutarate as co-substrates. Many of these enzymes also require ascorbate (vitamin C) as reducing agent.

Taurine dioxygenase (EC 1.14.11.17) is one such enzyme, which converts α-ketoglutarate to succinate and carbon dioxide, while also converting taurine and oxygen to sulfite and aminoacetaldehyde. Xanthine dioxygenase (EC 1.14.11.48) is another such enzyme, which converts α-ketoglutarate to succinate and carbon dioxide, while also converting xanthine and oxygen to urate.

With these dioxygenase enzymes, α-ketoglutarate is converted to succinate (succinic acid) which, unlike the succinic semialdehyde produced by KGDs, is not a substrate of any other enzyme in the process.

If amine donors other than glutamate or L-glutamate are used, the by-product can be reduced in amount using various additional methods. Those already known in the art include spontaneous decarboxylation of oxaloacetate (a by-product if aspartate is the amine donor) as described in U.S. Pat. No. 6,936,444 and, although not utilized to produce L-glufosinate, decarboxylation of pyruvate (a by-product if alanine is the amine donor) as described in U.S. Pat. No. 9,074,228. If certain amine donors other than glutamate or L-glutamate are used, decarboxylases can also be used to reduce the by-products. For example, branched-chain ketoacid decarboxylase can be used to decarboxylate 2-oxobutanoic acid, the by-product if 2-aminobutyric acid is the amine donor and alpha ketoisovaleric acid, the by-product if valine is the amine donor (Gocke, D., Nguyen, C. L., Pohl, M., Stillger, T., Walter, L. & Müller, M., Advanced Synthesis & Catalysis 2007, 349, 1425-1435). Other methods to reduce the amount of the by-product include reduction or oxidation of the carboxylic acid, aldehyde, or ketone, using either chemical or enzymatic means, for example, glycolaldehyde dehydrogenase can be used to catalyze the reduction of glycolaldehyde, the by-product if ethanolamine is the amine donor.

The effective levels of KG exposed to the TA enzyme could also be reduced by sequestering KG from the TA enzyme, and, optionally, the rest of the reaction mass. In one embodiment, a solid substrate with affinity for KG is present or added to the reaction vessel. This could include ion exchange resin, affinity resin, size exclusion resin, or other resin or substrate. In another embodiment, the reaction mass is removed from the presence of the TA enzyme and exposed to the solid substrate. For example, the reaction mass could be filtered from the main reaction vessel and applied to a resin that preferentially bound KG. The reaction mass containing reduced amounts of KG could be then re-exposed to the TA enzyme. Single, repeated, or continuous removal of KG from the reaction mass could shift the equilibrium of the reaction catalyzed by the TA enzyme toward L-glufosinate.

Several DAAO enzymes are known in the art and can be used in the methods described herein, as long as they are capable of accepting D-glufosinate as a substrate and provide an activity sufficient to level to drive the reaction. The DAAO enzymes useful in the methods described herein have an activity of equal to or greater than about 3 μmol/min*mg, greater than about 4 μmol/min*mg, or higher. A wild type enzyme can be used in the methods described herein as long as the enzyme has an activity level as set forth above. Such DAAO enzymes that can be used in the method include those from Rhodosporidium toruloides, Trigonopsis variabilis, Fusarium sp, Candida sp, Schizosasaccharomyces sp, Verticillium sp, Neolentinus lepideus, Trichoderma reesei, Trichosporon oleaginosus, and the like that have been modified to increase activity. Any DAAO enzyme can be used as a starting enzyme including those having sequences corresponding to Swissprot accession numbers P80324, Q99042, P00371, and P24552 or SPTREMBL numbers Q9HGY3 and Q9Y7N4 or GenBank numbers KZT28066.1, XP_006968548.1, and KLT40252.1. The DNA sequences which encode the DAAO may be selected from sequences set forth in EMBL accessions A56901, RGU60066, Z50019, SSDA04, D00809, AB042032, RCDAAOX, A81420, and SPCC1450, or may be codon optimized from the protein sequences indicated above for optimal expression in the chosen expression host(s). U.S. Pat. No. 8,227,228 describes DAAO enzymes from Candida intermedia. Such sequences are herein incorporated by reference. These enzymes can be modified for increased activity and used in the methods described herein.

Additional DAAO enzymes can be identified in a variety of ways, including sequence similarity and functional screens. The DAAO enzyme may be a mutant DAAO enzyme that is capable of accepting D-glufosinate as a substrate. In Hawkes et al., supra, a mutant DAAO based on the sequence from Rhodosporidium toruloides (consisting of the F58K and M213S mutations) has been shown to accept D-glufosinate as a substrate (Hawkes et al. (2011) Plant Biotechnol J. 9(3):301-14). Other DAAO enzymes can be similarly modified to accept D-glufosinate and have greater activity, i.e., the activity needed to drive the methods described herein. In the same manner, known DAAO enzymes may be improved by mutagenesis, and/or novel DAAO enzymes could be identified.

In some embodiments, mutant enzymes can be made and tested in the methods described herein. Mutant DAAO enzymes (e.g., from Rhodotorula gracilis) can include one mutation, two mutations, three mutations, or more than three mutations (e.g., four mutations, five mutations, six mutations, seven mutations, eight mutations, nine mutations, or ten mutations or more) at positions in the mutant sequence as compared to the wild type sequence. The mutant DAAO can optionally comprise mutations at positions 54, 56, 58, 213, and/or 238. In some embodiments, such mutants can comprise amino acid substitutions at positions 54 and 56 when compared with the wild type sequence. In other embodiments, such mutants can comprise amino acid substitutions at positions 54 and 58 when compared to the wild type sequence. In other embodiments, such mutants can include amino acid substitutions at positions 54, 213, and 238 when compared with the wild type sequence. In other embodiments, such mutants can include amino acid substitutions at positions 54, 56, 58, and 213 when compared with the wild type sequence.

Optionally, at position 54, the wild type asparagine may be replaced by Ala, Cys, Gly, IIe, Ser, Leu, or, more preferably, Thr or Val. For example, the mutant DAAO can comprise one of the following mutations at position 54: N54C, N54L, N54T, or N54V.

Optionally, at position 56, the wild type threonine can be replaced by Ala, Cys, Gly, IIe, Asn, Arg, Ser, Thr, Met, or Val. See, U.S. Pat. No. 7,939,709, which is incorporated herein by reference. For example, the mutant DAAO can comprise the T56M or the T56N mutation.

Additionally, at position 58, the wild type Phe can be replaced by Lys, Arg, Gln, Thr, Gly, Ser, Ala, Arg, Asn, or His. The mutant DAAO can optionally comprise one of the following mutations at position 58: F58A, F58G, F58H, F58K, F58N, F58Q, F58R, F58S, or F58T. In some embodiments, the mutant DAAO does not include a mutation at position 58.

Optionally, at position 213, the wild type methionine is replaced by Arg, Lys, Ser, Cys, Asn, or Ala. In some examples, the mutant DAAO can comprise the mutation M213S.

Optionally, at position 238, the wild type tyrosine is replaced by His, Ser, Cys, Asn, or Ala.

In some embodiments, the mutant DAAO can comprise one or more of the following combinations of mutations: F58K and M213S; N54T and T56M; N54V and F58Q; N54C and F58H; N54T and F58T; N54T and F58G; N54T and F58Q; N54T and F58A; N54L and F58R; N54V and F58R; N54V and F58N; N54V, T56N, F58H, and M213S; and/or N54V, F58Q, and M213S.

In one embodiment, the mutant DAAO comprises mutations in other DAAO enzymes in positions equivalent to positions 54, 56, 58, 213, and/or 238 of Rhodosporidium toruloides DAAO or Trigonopsis variabilis DAAO.

Other suitable D amino acid oxidases may be obtained preferably from fungal sources. Such DAAO enzymes can be identified and tested for use in the methods described herein. To determine if the enzyme will accept D-glufosinate as a substrate, an oxygen electrode assay (Hawkes, 2011, supra), colorimetric assay (Berneman A, Alves-Ferreira M, Coatnoan N, Chamond N, Minoprio P (2010) Medium/High Throughput D-Amino Acid Oxidase Colorimetric Method for Determination of D-Amino Acids. Application for Amino Acid Racemases. J Microbial Biochem Technol 2: 139-146), and/or direct measurement (via high performance liquid chromatography (HPLC), liquid chromatography mass spectrometry (LC-MS), or similar) of product formation can be employed.

The reaction catalyzed by the DAAO enzyme requires oxygen. Therefore, in one embodiment, aeration is a component of the DAAO reaction step. In some embodiments, oxygen, oxygen enriched air, an oxygen enriched gas stream, or air, is introduced to the reaction, either in the head space or by sparging gas through the reaction vessel, intermittently or continuously, to enhance the rate of reaction. Additionally, in other embodiments, optionally combined with sparging gas through the reaction vessel, a pressurized reactor may be used. That is, the reactor may be sealed and allowed to consume O₂. Using a sealed chamber would limit vapor emissions.

When a DAAO enzyme catalyzes the conversion of D-glufosinate to PPO, hydrogen peroxide (H₂O₂) evolves. This may be damaging to enzymes and other components of the biotransformation (e.g., products and/or substrates). Therefore, in one embodiment, an enzyme, such as catalase, can be used in addition to the DAAO enzyme to catalyze the elimination of hydrogen peroxide.

In some embodiments, hydrogen peroxide can be eliminated using catalyzed and non-catalyzed decomposition reactions. For example, hydrogen peroxide can be eliminated by a non-catalyzed decomposition reaction using increased heat and/or pH. Hydrogen peroxide can also be eliminated by a catalyzed decomposition reaction using, for example, transition metals and other agents, such as potassium iodide. In addition to eliminating hydrogen peroxide, the use of catalase also produces oxygen (O₂). The production of oxygen by catalase can aid in facilitating the conversion of D-glufosinate to PPO using the DAAO enzyme, as DAAO requires oxygen to function.

Other enzymes can be used to catalyze the conversion of D-glufosinate to PPO. For example, a DAAD enzyme that accepts D-glufosinate as a substrate can be used. It is recognized that in methods where a DAAD is used, the DAAD catalyzed reaction can include redox cofactor recycling. This involves oxidizing the reduced acceptor so that it can accept more electrons from D-glufosinate.

In one embodiment, chemical oxidative deamination, wherein an intermediate α-ketoacid is produced from the parent amino acid, can be used in the methods described herein to convert D-glufosinate to L-glufosinate. Chemical oxidative deamination involves the conversion of an amine group to a keto group with concomitant release of ammonia typically using metal ions such as those of copper or cobalt in an aqueous solution at temperatures between room temperature and the boiling point of the solution and at a pH in the range of about 4-about 10. See, for example, Ikawa and Snell (1954) J. Am. Chem. Soc. 76 (19): 4900-4902, herein incorporated by reference.

The substantially complete (greater than 70%, greater than 75%, greater than 80%, greater than 85%, greater than 90%, or greater than 95%) conversion of D-glufosinate to PPO can occur within 24 hours, within 18 hours, within 12 hours, within 8 hours, or less.

The second step of the method described herein involves the conversion of PPO to L-glufosinate using a transaminase (TA) enzyme. A TA with the required stereospecificity that accepts PPO as a substrate catalyzes the amination of PPO to L-glufosinate.

If the reaction is conducted as a two stage process where the D-glufosinate is substantially converted to PPO in the absence of amine donor and/or transaminase, starting amounts of PPO in the second stage typically range from 10 g/L to 140 g/L; 20 g/L to 140 g/L; or from 30 g/L to 140 g/L. If the reaction is conducted in a single stage process, the starting amounts of PPO are typically less than 1 g/L and the highest levels of PPO during the reaction are typically less than 25 g/L. The amine donor is initially present at between 1 and 50 fold molar excess over the starting amount of racemic glufosinate.

TAs useful in the methods described herein include the gabT transaminase from Escherichia coli (UniProt P22256), which has been shown to catalyze the desired reaction with PPO as a substrate (Bartsch et a. (1990) Appl Environ Microbiol. 56(1):7-12). Another enzyme has been evolved to catalyze the desired reaction at a higher rate using isopropylamine as an amine donor (Bhatia et a. (2004) Peptide Revolution: Genomics, Proteomics & Therapeutics, Proceedings of the Eighteenth American Peptide Symposium, Ed. Michael Chorev and Tomi K. Sawyer, Jul. 19-23, 2003, pp. 47-48). Additionally, TA enzymes from numerous microorganisms, such as Streptomyces hygroscopicus, Streptomyces viridochromogenes, Candida albicans, and others can be used in the practice of the methods described herein. In particular, see, for example, EP0249188, and U.S. Pat. No. 5,162,212, incorporated herein by reference. Where desired, the enzymes can be evolved by mutagenesis to increase their activities. Mutant TA enzymes can be selected for desired activity by the assays outlined in Schulz et al., Appl Environ Microbiol. (1990) Jan. 56(1):1-6, and/or by direct measurement of the products by HPLC, LC-MS, or similar products.

Additional TA enzymes for use in the methods can be identified by screening collections of TAs, such as those sold by Prozomix Limited (Northumberland, United Kingdom), SyncoZymes (Shanghai, China), Evocatal (Monheim am Rhein, Germany), Codexis (Redwood City, CA), or Abcam (Cambridge, United Kingdom) for the desired activity. Alternatively, sequence similarity can be used to identify novel TA enzymes. Finally, TA enzymes can also be identified from organisms capable of catalyzing the desired reaction.

The selection of an appropriate amine donor is important for an economical conversion of D-glufosinate to L-glufosinate. A variety of issues may be considered, including the cost of the donor, equilibrium thermodynamics, potential recovery of the donor, separation of the ketoacid product from the desired L-glufosinate, and others. Consequently, TA enzymes that accept several different amine donors can be used, including low cost amine donors such as L-aspartate or racemic aspartate, L-glutamate or racemic glutamate, L-alanine or racemic alanine, L-phenylethylamine or racemic phenylalanine, L-glycine or racemic glycine, L-lysine or racemic lysine, L-valine or racemic valine, L-serine or racemic serine, L-glutamine or racemic glutamine, isopropylamine, sec-butylamine, ethanolamine, 2-aminobutyric acid, and diaminoproprionic acid. In some embodiments, the amine donor is not aspartate or aspartic acid (e.g., L-aspartic acid, D-aspartic acid, or racemic D,L-aspartic acid).

A wild type TA that accepts a desired amine donor can be identified, or a TA that does not normally accept a desired amine donor can be evolved to accept the desired substrate. Optionally, the transaminase is not an aspartate transaminase. Optionally, the transaminase is not 4-amino-butyrate: 2-ketoglutarate transaminase. In some embodiments, the transaminase is not a combination enzyme system that includes a PPT-specific transaminase and glutamate:oxaloacetate transaminase.

As indicated in a third step of the method to produce L-glufosinate, the alpha keto by-product produced is reduced driving the reaction to complete or substantially complete conversion of the PPO to L-glufosinate. As indicated, the means for reducing the by-product may vary depending on the amine donor.

Using the methods described herein, substantially complete conversion of PPO to L-glufosinate may occur within 48 hours, within 24 hours, within 12 hours, within 8 hours, or within 4 hours. Substantially complete, in this context, means that the conversion of PPO to L-glufosinate is greater than about 90%, greater than about 95%, greater than about 96%, greater than about 97%, greater than about 98%, greater than about 99%, or all detectable PPO is converted.

If the reaction occurs in a single container or vessel, the TA enzyme can be added with the DAAO enzyme or added at a later time, e.g., after the DAAO enzyme has been allowed to catalyze some or substantially all of the oxidative deamination.

Enzymes can be added to the reaction by a number of methods. One approach is to express the enzyme(s) in microorganism(s) such as E. coli, S. cerevisiae, P. pastoris, and others, and to add the whole cells to the reactions as whole cell biocatalysts. Another approach is to express the enzyme(s), lyse the microorganisms, and add the cell lysate. Yet another approach is to purify, or partially purify, the enzyme(s) from a lysate and add pure or partially pure enzyme(s) to the reaction. If multiple enzymes are required for a reaction, the enzymes can be expressed in one or several microorganisms, including expressing all enzymes within a single microorganism.

A further approach, which can be combined with the above approaches, is to immobilize enzyme(s) to a support (exemplary strategies are outlined in Datta et al. (2013) 3 Biotech. Feb; 3(1): 1-9). Not intending to be limiting, enzymes, either singly or in combination, can, for example, be adsorbed to, or covalently or non-covalently attached to, or entrapped within, natural or synthetic polymers or inorganic supports, including aggregates of the enzyme(s) themselves. Once immobilized, the enzyme(s) and support can be dispersed into bulk solution or packed into beds, columns, or any number of similar approaches to interacting reaction solution with the enzymes. Since aeration is important for the DAAO reaction envisioned here, bubble columns or similar may be used for enzyme immobilization. As examples, reaction mixture can be flowed through a column of immobilized enzymes (flow reaction), added to a fixed bed or column of immobilized enzymes, allowed to react, and either removed from the bottom or top of the reaction vessel (plug flow), or added to dispersed immobilized enzymes and allowed to react then the immobilized enzymes removed by filtration, centrifugation, or similar (batch). Thus, any method for immobilization of the enzymes may be employed in the methods described herein.

The DAAO, TA, and/or other reactions can occur in a buffer. Exemplary buffers commonly used in biotransformation reactions include Tris, phosphate, or any of Good's buffers, such as 2-(N-morpholino)ethanesulfonic acid (MES); N-(2-Acetamido)iminodiacetic acid (ADA); piperazine-N,N′-bis(2-ethanesulfonic acid) (PIPES); N-(2-Acetamido)-2-aminoethanesulfonic acid (ACES); β-Hydroxy-4-morpholinepropanesulfonic acid (MOPSO); cholamine chloride; 3-(N-morpholino)propanesulfonic acid (MOPS); N,N-Bis(2-hydroxyethyl)-2-aminoethanesulfonic acid (BES); 2-[[1,3-dihydroxy-2-(hydroxymethyl)propan-2-yl]amino]ethanesulfonic acid (TES); 4-(2-hydroxyethyl)-1-piperazineethanesulfonic acid (HEPES); 3-(Bis(2-hydroxyethyl)amino)-2-hydroxypropane-1-sulfonic acid (DIPSO); acetamidoglycine, 3-(N-Tris(hydroxymethyl)methylamino(-2-hydroxypropanesulfonic acid (TAPSO); Piperazine-N,N′-bis(2-hydroxypropanesulfonic acid) (POPSO); 4-(2-Hydroxyethyl)piperazine-1-(2-hydroxypropanesulfonic acid) (HEPPSO); 3-[4-(2-Hydroxyethyl)-1-piperazinyl]propanesulfonic acid (HEPPS); tricine; glycinamide; bicine; or 3-[[1,3-dihydroxy-2-(hydroxymethyl)propan-2-yl]amino]propane-1-sulfonic acid (TAPS). Additional exemplary buffer recipes can be found in Whittall, J. and Sutton, P. W. (eds) (2012) Front Matter, in Practical Methods for Biocatalysis and Biotransformations 2, John Wiley & Sons, Ltd, Chichester, UK. In some embodiments, ammonium can act as a buffer. One or more organic solvents can also be added to the reaction.

Surprisingly, the DAAO, TA, and/or other reactions can occur with no or low levels (less than 1 mM) of buffer added (other than ammonium that may optionally be present due to addition of racemic glufosinate ammonium). In particular, immobilized DAAO, TA, and KGD enzymes may be stable and active in the presence of less than 1 mM phosphate buffer and with no other buffer except any ammonium present due to the addition of racemic glufosinate ammonium.

The racemic glufosinate starting material can be provided in a number of forms. Various salts of racemic glufosinate, such as ammonium and hydrochloride, or the zwitterion, can be used. The racemic glufosinate may be in the form of a solid powder (such as a powder of greater than 80%, 85%, 90%, or 95% purity) or an aqueous solution (such as a roughly 50% solution of racemic glufosinate).

In some embodiments, the reaction occurs within a defined pH range, which can be between pH 4 to pH 10 (e.g., between pH 6 and pH 9, such as approximately pH 7.5 to pH 8).

In some embodiments, the reaction occurs at a defined temperature. The temperature can be kept at a point between room temperature and the boiling point of the solvent, most typically between room temperature and 50° C.

Additional steps to further purify the L-glufosinate can be added. Such further purification and isolation methods include ion exchange, extraction, salt formation, crystallization and filtration; each may be used multiple times or in suitable combination. Enzymes can be removed by simple filtration if supported, or if free in solution by the use of ultrafiltration, the use of absorbants like celite, cellulose or carbon, or denaturation via various techniques known to those skilled in the art.

Ion exchange processes effect separation by selective adsorption of solutes onto resins chosen for this purpose. Because products and impurities must be dissolved in a single solution prior to adsorption, concentration of the purified product stream by evaporation or distillation prior to isolation is usually required. Examples of the use of ion exchange for purification are described by Schultz et al., EP0249188(A2), U.S. Pat. No. 9,834,802, and International PCT Patent Application PCT/US2018/042503.

Purification may be achieved by the formation of an insoluble salt of L-glufosinate by the addition of a suitable acid, including hydrochloric acid, sulfuric acid, phosphoric acid, nitric acid, acetic acid and the like. Similarly, the purification may be achieved by the addition of a suitable base to form an insoluble salt. Useful bases include hydroxides, carbonates, sulfates and phosphates of alkali metals or hydroxides, carbonates, sulfates and phosphates of alkali earth metals. Other bases which contain nitrogen may be used, including ammonia, hydroxylamine, isopropylamine, triethylamine, tributylamine, pyridine, 2-picoline, 3-picoline, 4-picoline, 2,4-lutidine, 2,6-lutidine, morpholine, N-methymorpholine, 1,8-diazabicyclo[5.4.0]undec-7-ene, and dimethylethanolamine. It may be advantageous to concentrate the mixture or to add a solvent (or both) to maximize yield and optimize purity of the desired salt. Solvents suitable for this purpose include those in which the solubility of the desired salt is very low (such solvents are often called “anti-solvents”). Salts of L-glufosinate can be transformed into forms of glufosinate suitable for formulation by standard methods known to those skilled in the art. Alternatively, the L-glufosinate can be isolated as a zwitterion.

U.S. Pat. No. 9,255,115 B2 describes how the hydrochloric acid salt of L-glufosinate can be converted to the zwitterionic form with a base such as sodium hydroxide or sodium methoxide and then crystallized from aqueous alcohol solvent to afford L-glufosinate in relatively high purity. This method has the advantage of producing crystalline L-glufosinate that is not hygroscopic and therefore maintains a higher purity compared to amorphous L-glufosinate when exposed to humidity over time.

Other salts of L-glufosinate are known in the art. U.S. Pat. Nos. 5,767,309 and 5,869,668 teach the use of chiral alkaloid bases to form diastereomeric salts with racemic glufosinate. Purification is achieved because the salt of L-glufosinate precipitates from solution in much larger quantity than the corresponding salt of D-glufosinate. Therefore, this method could be used with the methods described herein to obtain L-glufosinate with high enantiomeric excess, if desired.

Optionally, purification may be achieved by first crystallizing one or more impurities, removing the impurities by filtration and then further purifying L-glufosinate from the resulting filtrate by forming a salt as previously described. This is advantageous if unreacted amine donor can be partially or completely isolated and used in subsequent reactions. Similarly, unreacted PPO that is partially or completely isolated may be recycled for use in subsequent reactions.

Extraction may be used to purify the product. DE 3920570 C2 describes a process in which excess glutamic acid (used as the amine donor) is precipitated by adjusting the solution pH to 3.7 to 4.2 with sulfuric acid. After filtering the glutamic acid, the filtrate pH is lowered to 1-2 whereupon other impurities are extracted into a solvent. After extraction and concentration, ammonia is added to the aqueous solution to a pH of 5-7 whereupon ammonium sulfate precipitates. The ammonium sulfate is removed by filtration and the resulting filtrate is concentrated to afford the ammonium salt of L-glufosinate.

Isolation of L-glufosinate or its salts may be desirable, for example, for the purpose of shipping solids to the location of formulation or use. Typical industrial methods of isolation may be used, for example, filtration, centrifugation, etc. Isolated product often requires the removal of water, volatile impurities and solvents (if present) and typical industrial drying equipment may be used for this purpose. Examples of such equipment include ovens, rotating drum dryers, agitated dryers, etc. In some cases, it may be advantageous to use a spray dryer.

It is not necessary to produce a solid product after purification. This may be advantageous if the formulation of L-glufosinate is to occur at the same site used for L-glufosinate production. L-glufosinate and many of its salts are readily soluble in water, and water is a convenient liquid to use for formulating products. For example, the amine donor is isolated by filtration and the resulting filtrate is concentrated by distillation. The pH of the filtrate may be adjusted to a desirable value and the resulting solution may be used as is or blended with formulation ingredients. In another example, a slurry of L-glufosinate or one of its salts may be prepared as described above and isolated by filtration. The solid could be dissolved directly on the filter by adding water or a suitable solvent to obtain a solution of L-glufosinate.

### II. Compositions

Also described herein are compositions comprising the reaction products described above. In some embodiments, the composition substantially includes L-glufosinate and acceptable cationic or anionic salt forms such as the hydrochloride, ammonium, or isopropylammonium salts. In some embodiments, the composition comprises a mixture of L-glufosinate, PPO, and SSA. In some other embodiments, the compositions comprises a mixture of L-glufosinate and SSA.

The compositions described herein are useful for application to a field of crop plants for the prevention or control of weeds. The composition may be formulated as a liquid for spraying on a field. The L-glufosinate is provided in the composition in effective amounts. As used herein, effective amount means from about 10 grams active ingredient per hectare to about 1,500 grams active ingredient per hectare, e.g., from about 50 grams to about 400 grams or from about 100 grams to about 350 grams. In some embodiments, the active ingredient is L-glufosinate. For example, the amount of L-glufosinate in the composition can be about 10 grams, about 50 grams, about 100 grams, about 150 grams, about 200 grams, about 250 grams, about 300 grams, about 350 grams, about 400 grams, about 500 grams, about 550 grams, about 600 grams, about 650 grams, about 700 grams, about 750 grams, about 800 grams, about 850 grams, about 900 grams, about 950 grams, about 1,000 grams, about 1,050 grams, about 1,100 grams, about 1,150 grams, about 1,200 grams, about 1,250 grams, about 1,300 grams, about 1,350 grams, about 1,400 grams, about 1,450 grams, or about 1,500 grams L-glufosinate per hectare.

The herbicidal compositions (including concentrates which require dilution prior to application to the plants) described herein contain L-glufosinate (i.e., the active ingredient), optionally some residual D-glufosinate and/or PPO, and one or more adjuvant components in liquid or solid form.

The compositions are prepared by admixing the active ingredient with one or more adjuvants, such as diluents, extenders, carriers, surfactants, organic solvents, humectants, or conditioning agents, to provide a composition in the form of a finely-divided particulate solid, pellet, solution, dispersion, or emulsion. Thus, the active ingredient can be used with an adjuvant, such as a finely-divided solid, a liquid of organic origin, water, a wetting agent, a dispersing agent, an emulsifying agent, or any suitable combination of these. From the viewpoint of economy and convenience, water is the preferred diluent. However, not all the compounds are resistant to hydrolysis and in some cases this may dictate the use of non-aqueous solvent media, as understood by those of skill in the art.

Optionally, one or more additional components can be added to the composition to produce a formulated herbicidal composition. Such formulated compositions can include L-glufosinate, carriers (e.g., diluents and/or solvents), and other components. The formulated composition includes an effective amount of L-glufosinate. Optionally, the L-glufosinate can be present in the form of L-glufosinate ammonium. The L-glufosinate ammonium can be present in an amount ranging from 10% to 30% by weight of the formulated composition. For example, the L-glufosinate ammonium can be present in an amount of 10%, 12%, 14%, 16%, 18%, 20%, 22%, 24%, 26%, 28%, or 30% by weight of the formulated composition. Optionally, the L-glufosinate ammonium is present in an amount of 12.25% or of 24.5% by weight of the formulated composition.

In some examples, the formulated composition can include one or more surfactants. A suitable surfactant for use in the formulated composition includes sodium alkyl ether sulfate. The surfactant can be present in an amount from 10% to 40% by weight of the formulated composition. For example, the surfactant can be present in an amount of 10%, 12%, 14%, 16%, 18%, 20%, 22%, 24%, 26%, 28%, 30%, 32%, 34%, 36%, 38%, or 40% by weight of the formulated composition. Optionally, the sodium alkyl ether sulfate is present in an amount of 11.05%, 15.8%, 22.1%, or 31.6% by weight of the formulated composition.

The formulated composition can optionally include one or more solvents (e.g., organic solvents). Optionally, the solvent can be 1-methoxy-2-propanol, dipropylene glycol, ethylene glycol, propylene glycol, and mixtures thereof. The one or more solvents can be present in an amount ranging from 0.5% to 20% by weight of the formulated composition. For example, the total amount of solvents in the composition can be present in an amount of 0.5% to 18%, 5% to 15%, or 7.5% to 10% by weight of the formulated composition.

Optionally, the solvent includes a combination of two solvents. For example, the solvents in the formulation can include 1-methoxy-2-propanol and dipropylene glycol. The 1-methoxy-2-propanol can be present, for example, in an amount of 0.5% to 2% by weight of the formulated composition. For example, the 1-methoxy-2-propanol can be present in the amount of 0.5%, 0.6%, 0.7%, 0.8%, 0.9%, 1.0%, 1.1%1.2%, 1.3%, 1.4%, 1.5%, 1.6%, 1.7%, 1.8%, 1.9%, or 2.0% by weight of the formulated composition. Optionally, the 1-methoxy-2-propanol is present in an amount of 0.5% or 1.0% by weight of the formulated composition. The dipropylene glycol can be present in an amount of from 4% to 18% by weight of the formulated composition. For example, the dipropylene glycol can be present in an amount of 4%, 6%, 8%, 10%, 12%, 14%, 16%, or 18% by weight of the formulated composition. Optionally, the dipropylene glycol is present in an amount of 4.3% or 8.6% by weight of the formulated composition.

The formulated composition can also include one or more polysaccharide humectants. Examples of suitable polysaccharide humectants include, for example, alkyl polysaccharides, pentoses, high fructose corn syrup, sorbitol, and molasses. The polysaccharide humectant, such as alkyl polysaccharide, can be present in the formulated composition in an amount ranging from 4% to 20% by weight of the formulated composition. For example, the total amount of polysaccharide humectant in the composition can be present in an amount of 4% to 18%, 4.5% to 15%, or 5% to 10% by weight of the formulated composition. In some examples, the total amount of polysaccharide humectant, such as the alkyl polysaccharide, present in the formulated composition can be 4%, 5%, 6%, 7%, 8%, 9%, 10%, 11%, 12%, 13%, 14%, 15%, 16%, 17%, or 18%. Optionally, the alkyl polysaccharide can be present in an amount of 3.2%, 4.9%, 6.2%, or 9.8% by weight of the formulated composition.

A diluent can also be included in the formulated composition. Suitable diluents include water and other aqueous components. Optionally, the diluents are present in an amount necessary to produce compositions ready for packaging or for use.

In one example, the formulated composition includes L-glufosinate ammonium in an amount of 12.25% by weight of the formulation; sodium alkyl ether sulfate in an amount of 31.6% by weight of the formulation; 1-methoxy-2-propanol in an amount of 1% by weight of the formulation; dipropylene glycol in an amount of 8.6% by weight of the formulation; and alkyl polysaccharide in an amount of 9.8% by weight of the formulation.

In another example, the formulated composition includes L-glufosinate ammonium in an amount of 24.5% by weight of the formulation; sodium alkyl ether sulfate in an amount of 31.6% by weight of the formulation; 1-methoxy-2-propanol in an amount of 1% by weight of the formulation; dipropylene glycol in an amount of 8.6% by weight of the formulation; and alkyl polysaccharide in an amount of 9.8% by weight of the formulation.

In another example, the formulated composition includes L-glufosinate ammonium in an amount of 12.25% by weight of the formulation; sodium alkyl ether sulfate in an amount of 15.8% by weight of the formulation; 1-methoxy-2-propanol in an amount of 0.5% by weight of the formulation; dipropylene glycol in an amount of 4.3% by weight of the formulation; and alkyl polysaccharide in an amount of 4.9% by weight of the formulation.

In another example, the formulated composition includes L-glufosinate ammonium in an amount of 24.5% by weight of the formulation; sodium alkyl ether sulfate in an amount of 22.1% by weight of the formulation; 1-methoxy-2-propanol in an amount of 1% by weight of the formulation; and alkyl polysaccharide in an amount of 6.2% by weight of the formulation.

In another example, the formulated composition includes L-glufosinate ammonium in an amount of 24.5% by weight of the formulation; sodium alkyl ether sulfate in an amount of 22.1% by weight of the formulation; propylene glycol in an amount of 15% by weight of the formulation; and alkyl polysaccharide in an amount of 6.2% by weight of the formulation.

In another example, the formulated composition includes L-glufosinate ammonium in an amount of 12.25% by weight of the formulation; sodium alkyl ether sulfate in an amount of 22.1% by weight of the formulation; 1-methoxy-2-propanol in an amount of 1% by weight of the formulation; and alkyl polysaccharide in an amount of 6.2% by weight of the formulation.

In another example, the formulated composition includes L-glufosinate ammonium in an amount of 12.25% by weight of the formulation; sodium alkyl ether sulfate in an amount of 11.05% by weight of the formulation; 1-methoxy-2-propanol in an amount of 0.5% by weight of the formulation; and alkyl polysaccharide in an amount of 3.1% by weight of the formulation.

Further components suitable for use in the formulated compositions provided herein are described in U.S. Pat. Nos. 4,692,181 and 5,258,358, both of which are incorporated by reference herein in their entireties.

The herbicidal compositions described herein, particularly liquids and soluble powders, can contain as further adjuvant components one or more surface-active agents in amounts sufficient to render a given composition readily dispersible in water or in oil. The incorporation of a surface-active agent into the compositions greatly enhances their efficacy. Surface-active agent, as used herein, includes wetting agents, dispersing agents, suspending agents, and emulsifying agents are included therein. Anionic, cationic, and non-ionic agents can be used with equal facility.

Suitable wetting agents include alkyl benzene and alkyl naphthalene sulfonates, sulfated fatty alcohols, amines or acid amides, long chain acid esters of sodium isothionate, esters of sodium sulfosuccinate, sulfated or sulfonated fatty acid esters petroleum solfonates, sulfonated vegetable oils, ditertiary acetylenic glycols, polyoxyethylene derivatives of alkylphenols (particularly isooctylphenol and nonylphenol), and polyoxethylene derivatives of the mono-higher fatty acid esters of hexitol anhydrides (e.g. sorbitan). Exemplary dispersants include methyl cellulose, polyvinyl alcohol, sodium lignin sulfonates, polymeric alkyl naphthalene sulfonates, sodium naphthalene sulfonate, polymethylene bisnaphthalenesulfonate, and sodium N-methyl-N-(long chain acid) laurates.

Water-dispersible powder compositions can be made containing one or more active ingredients, an inert solid extender, and one or more wetting and dispersing agents. The inert solid extenders are usually of mineral origin, such as the natural clays, diatomaceous earth, and synthetic minerals derived from silica and the like. Examples of such extenders include kaolinites, attapulgite clay, and synthetic magnesium silicate. Water-dispersible powders described herein can optionally contain from about 5 to about 95 parts by weight of active ingredient (e.g., from about 15 to 30 parts by weight of active ingredient), from about 0.25 to 25 parts by weight of wetting agent, from about 0.25 to 25 parts by weight of dispersant, and from 4.5 to about 94.5 parts by weight of inert solid extender, all parts being by weight of the total composition. Where required, from about 0.1 to 2.0 parts by weight of the solid inert extender can be replaced by a corrosion inhibitor or anti-foaming agent or both.

Aqueous suspensions can be prepared by dissolution or by mixing together and grinding an aqueous slurry of a water-insoluble active ingredient in the presence of a dispersing agent to obtain a concentrated slurry of very finely-divided particles. The resulting concentrated aqueous suspension is characterized by its extremely small particle size, so that when diluted and sprayed, coverage is very uniform.

Emulsifiable oils are usually solutions of active ingredient in water-immiscible or partially water-immiscible solvents together with a surface active agent. Suitable solvents for the active ingredient described herein include hydrocarbons and water-immiscible ethers, esters, or ketones. The emulsifiable oil compositions generally contain from about 5 to 95 parts active ingredient, about 1 to 50 parts surface active agent, and about 4 to 94 parts solvent, all parts being by weight based on the total weight of emulsifiable oil.

Compositions described herein can also contain other additaments, for example, fertilizers, phytotoxicants and plant growth regulants, pesticides, and the like used as adjuvants or in combination with any of the above-described adjuvants. The compositions described herein can also be admixed with the other materials, e.g., fertilizers, other phytotoxicants, etc., and applied in a single application.

In each of the formulation types described herein, e.g., liquid and solid formulations, the concentration of the active ingredients are the same.

It is recognized that the herbicidal compositions can be used in combination with other herbicides. The herbicidal compositions described herein are often applied in conjunction with one or more other herbicides to control a wider variety of undesirable vegetation. When used in conjunction with other herbicides, the presently claimed compounds can be formulated with the other herbicide or herbicides, tank mixed with the other herbicide or herbicides or applied sequentially with the other herbicide or herbicides. Some of the herbicides that can be employed in conjunction with the compounds described herein include: amide herbicides such as allidochlor, beflubutamid, benzadox, benzipram, bromobutide, cafenstrole, CDEA, chlorthiamid, cyprazole, dimethenamid, dimethenamid-P, diphenamid, epronaz, etnipromid, fentrazamide, flupoxam, fomesafen, halosafen, isocarbamid, isoxaben, napropamide, naptalam, pethoxamid, propyzamide, quinonamid and tebutam; anilide herbicides such as chloranocryl, cisanilide, clomeprop, cypromid, diflufenican, etobenzanid, fenasulam, flufenacet, flufenican, mefenacet, mefluidide, metamifop, monalide, naproanilide, pentanochlor, picolinafen and propanil; arylalanine herbicides such as benzoylprop, flamprop and flamprop-M; chloroacetanilide herbicides such as acetochlor, alachlor, butachlor, butenachlor, delachlor, diethatyl, dimethachlor, metazachlor, metolachlor, S-metolachlor, pretilachlor, propachlor, propisochlor, prynachlor, terbuchlor, thenylchlor and xylachlor; sulfonanilide herbicides such as benzofluor, perfluidone, pyrimisulfan and profluazol; sulfonamide herbicides such as asulam, carbasulam, fenasulam and oryzalin; antibiotic herbicides such as bilanafos; benzoic acid herbicides such as chloramben, dicamba, 2,3,6-TBA and tricamba; pyrimidinyloxybenzoic acid herbicides such as bispyribac and pyriminobac; pyrimidinylthiobenzoic acid herbicides such as pyrithiobac; phthalic acid herbicides such as chlorthal; picolinic acid herbicides such as aminopyralid, clopyralid and picloram; quinolinecarboxylic acid herbicides such as quinclorac and quinmerac; arsenical herbicides such as cacodylic acid, CMA, DSMA, hexaflurate, MAA, MAMA, MSMA, potassium arsenite and sodium arsenite; benzoylcyclohexanedione herbicides such as mesotrione, sulcotrione, tefuryltrione and tembotrione; benzofuranyl alkylsulfonate herbicides such as benfuresate and ethofumesate; carbamate herbicides such as asulam, carboxazole chlorprocarb, dichlormate, fenasulam, karbutilate and terbucarb; carbanilate herbicides such as barban, BCPC, carbasulam, carbetamide, CEPC, chlorbufam, chlorpropham, CPPC, desmedipham, phenisopham, phenmedipham, phenmedipham-ethyl, propham and swep; cyclohexene oxime herbicides such as alloxydim, butroxydim, clethodim, cloproxydim, cycloxydim, profoxydim, sethoxydim, tepraloxydim and tralkoxydim; cyclopropylisoxazole herbicides such as isoxachlortole and isoxaflutole; dicarboximide herbicides such as benzfendizone, cinidon-ethyl, flumezin, flumiclorac, flumioxazin and flumipropyn; dinitroaniline herbicides such as benfluralin, butralin, dinitramine, ethalfluralin, fluchloralin, isopropalin, methalpropalin, nitralin, oryzalin, pendimethalin, prodiamine, profluralin and trifluralin; dinitrophenol herbicides such as dinofenate, dinoprop, dinosam, dinoseb, dinoterb, DNOC, etinofen and medinoterb; diphenyl ether herbicides such as ethoxyfen; nitrophenyl ether herbicides such as acifluorfen, aclonifen, bifenox, chlomethoxyfen, chlomitrofen, etnipromid, fluorodifen, fluoroglycofen, fluoronitrofen, fomesafen, furyloxyfen, halosafen, lactofen, nitrofen, nitrofluorfen and oxyfluorfen; dithiocarbamate herbicides such as dazomet and metam; halogenated aliphatic herbicides such as alorac, chloropon, dalapon, flupropanate, hexachloroacetone, iodomethane, methyl bromide, monochloroacetic acid, SMA and TCA; imidazolinone herbicides such as imazamethabenz, imazamox, imazapic, imazapyr, imazaquin and imazethapyr; inorganic herbicides such as ammonium sulfamate, borax, calcium chlorate, copper sulfate, ferrous sulfate, potassium azide, potassium cyanate, sodium azide, sodium chlorate and sulfuric acid; nitrile herbicides such as bromobonil, bromoxynil, chloroxynil, dichlobenil, iodobonil, ioxynil and pyraclonil; organophosphorus herbicides such as amiprofos-methyl, anilofos, bensulide, bilanafos, butamifos, 2,4-DEP, DMPA, EBEP, fosamine, glyphosate and piperophos; phenoxy herbicides such as bromofenoxim, clomeprop, 2,4-DEB, 2,4-DEP, difenopenten, disul, erbon, etnipromid, fenteracol and trifopsime; phenoxyacetic herbicides such as 4-CPA, 2,4-D, 3,4-DA, MCPA, MCPA-thioethyl and 2,4,5-T; phenoxybutyric herbicides such as 4-CPB, 2,4-DB, 3,4-DB, MCPB and 2,4,5-TB; phenoxypropionic herbicides such as cloprop, 4-CPP, dichlorprop, dichlorprop-P, 3,4-DP, fenoprop, mecoprop and mecoprop-P; aryloxyphenoxypropionic herbicides such as chlorazifop, clodinafop, clofop, cyhalofop, diclofop, fenoxaprop, fenoxaprop-P, fenthiaprop, fluazifop, fluazifop-P, haloxyfop, haloxyfop-P, isoxapyrifop, metamifop, propaquizafop, quizalofop, quizalofop-P and trifop; phenylenediamine herbicides such as dinitramine and prodiamine; pyrazolyl herbicides such as benzofenap, pyrazolynate, pyrasulfotole, pyrazoxyfen, pyroxasulfone and topramezone; pyrazolylphenyl herbicides such as fluazolate and pyraflufen; pyridazine herbicides such as credazine, pyridafol and pyridate; pyridazinone herbicides such as brompyrazon, chloridazon, dimidazon, flufenpyr, metflurazon, norflurazon, oxapyrazon and pydanon; pyridine herbicides such as aminopyralid, cliodinate, clopyralid, dithiopyr, fluroxypyr, haloxydine, picloram, picolinafen, pyriclor, thiazopyr and triclopyr; pyrimidinediamine herbicides such as iprymidam and tioclorim; quaternary ammonium herbicides such as cyperquat, diethamquat, difenzoquat, diquat, morfamquat and paraquat; thiocarbamate herbicides such as butylate, cycloate, di-allate, EPTC, esprocarb, ethiolate, isopolinate, methiobencarb, molinate, orbencarb, pebulate, prosulfocarb, pyributicarb, sulfallate, thiobencarb, tiocarbazil, tri-allate and vemolate; thiocarbonate herbicides such as dimexano, EXD and proxan; thiourea herbicides such as methiuron; triazine herbicides such as dipropetryn, triaziflam and trihydroxytriazine; chlorotriazine herbicides such as atrazine, chlorazine, cyanazine, cyprazine, eglinazine, ipazine, mesoprazine, procyazine, proglinazine, propazine, sebuthylazine, simazine, terbuthylazine and trietazine; methoxytriazine herbicides such as atraton, methometon, prometon, secbumeton, simeton and terbumeton; methylthiotriazine herbicides such as ametryn, aziprotryne, cyanatryn, desmetryn, dimethametryn, methoprotryne, prometryn, simetryn and terbutryn; triazinone herbicides such as ametridione, amibuzin, hexazinone, isomethiozin, metamitron and metribuzin; triazole herbicides such as amitrole, cafenstrole, epronaz and flupoxam; triazolone herbicides such as amicarbazone, bencarbazone, carfentrazone, flucarbazone, propoxycarbazone, sulfentrazone and thiencarbazone-methyl; triazolopyrimidine herbicides such as cloransulam, diclosulam, florasulam, flumetsulam, metosulam, penoxsulam and pyroxsulam; uracil herbicides such as butafenacil, bromacil, flupropacil, isocil, lenacil and terbacil; 3-phenyluracils; urea herbicides such as benzthiazuron, cumyluron, cycluron, dichloralurea, diflufenzopyr, isonoruron, isouron, methabenzthiazuron, monisouron and noruron; phenylurea herbicides such as anisuron, buturon, chlorbromuron, chloreturon, chlorotoluron, chloroxuron, daimuron, difenoxuron, dimefuron, diuron, fenuron, fluometuron, fluothiuron, isoproturon, linuron, methiuron, methyldymron, metobenzuron, metobromuron, metoxuron, monolinuron, monuron, neburon, parafluron, phenobenzuron, siduron, tetrafluron and thidiazuron; pyrimidinylsulfonylurea herbicides such as amidosulfuron, azimsulfuron, bensulfuron, chlorimuron, cyclosulfamuron, ethoxysulfuron, flazasulfuron, flucetosulfuron, flupyrsulfuron, foramsulfuron, halosulfuron, imazosulfuron, mesosulfuron, nicosulfuron, orthosulfamuron, oxasulfuron, primisulfuron, pyrazosulfuron, rimsulfuron, sulfometuron, sulfosulfuron and trifloxysulfuron; triazinylsulfonylurea herbicides such as chlorsulfuron, cinosulfuron, ethametsulfuron, iodosulfuron, metsulfuron, prosulfuron, thifensulfuron, triasulfuron, tribenuron, triflusulfuron and tritosulfuron; thiadiazolylurea herbicides such as buthiuron, ethidimuron, tebuthiuron, thiazafluron and thidiazuron; and unclassified herbicides such as acrolein, allyl alcohol, aminocyclopyrachlor, azafenidin, benazolin, bentazone, benzobicyclon, buthidazole, calcium cyanamide, cambendichlor, chlorfenac, chlorfenprop, chlorflurazole, chlorflurenol, cinmethylin, clomazone, CPMF, cresol, ortho-dichlorobenzene, dimepiperate, endothal, fluoromidine, fluridone, flurochloridone, flurtamone, fluthiacet, indanofan, methazole, methyl isothiocyanate, nipyraclofen, OCH, oxadiargyl, oxadiazon, oxaziclomefone, pentachlorophenol, pentoxazone, phenylmercury acetate, pinoxaden, prosulfalin, pyribenzoxim, pyriftalid, quinoclamine, rhodethanil, sulglycapin, thidiazimin, tridiphane, trimeturon, tripropindan and tritac. The herbicidal compositions described herein can, further, be used in conjunction with glyphosate or 2,4-D on glyphosate-tolerant or 2,4-D-tolerant crops. It is generally preferred to use the compositions described herein in combination with herbicides that are selective for the crop being treated and which complement the spectrum of weeds controlled by these compositions at the application rate employed. It is further generally preferred to apply the compositions described herein and other complementary herbicides at the same time, either as a combination formulation or as a tank mix.

### III. Methods of Use of L-Glufosinate Compositions

The compositions described herein can be used in methods for selectively controlling weeds in a field or any other area, including, for example, a railway, lawn, golf course, and others where the control of weeds is desired. Optionally, the field or other area can contain a crop of planted seeds or crops that are resistant to glufosinate. The methods can include applying an effective amount of a composition comprising L-glufosinate as described herein to the field.

The compositions described herein are useful for application to a field of crop plants for the prevention or control of weeds. The composition may be formulated as a liquid for spraying on a field. The L-glufosinate is provided in the composition in effective amounts. As used herein, effective amount means from about 10 grams active ingredient per hectare to about 1,500 grams active ingredient per hectare, e.g., from about 50 grams to about 400 grams or from about 100 grams to about 350 grams. In some embodiments, the active ingredient is L-glufosinate. For example, the amount of L-glufosinate in the composition can be about 10 grams, about 50 grams, about 100 grams, about 150 grams, about 200 grams, about 250 grams, about 300 grams, about 350 grams, about 400 grams, about 500 grams, about 550 grams, about 600 grams, about 650 grams, about 700 grams, about 750 grams, about 800 grams, about 850 grams, about 900 grams, about 950 grams, about 1,000 grams, about 1,050 grams, about 1,100 grams, about 1,150 grams, about 1,200 grams, about 1,250 grams, about 1,300 grams, about 1,350 grams, about 1,400 grams, about 1,450 grams, or about 1,500 grams L-glufosinate per hectare.

### IV. Exemplary Embodiments

Non-limiting embodiments include:
1. A method for making L-glufosinate, comprising: reacting D-glufosinate with a D-amino acid oxidase (DAAO) enzyme to form PPO (2-oxo-4-(hydroxy(methyl)phosphinoyl)butyric acid) while aerating in an oxidation step; aminating the PPO by a transaminase (TA) enzyme, using an amine group from one or more amine donors to form L-glufosinate and an alpha ketoacid by-product or ketone by-product in an amination step; and reducing an amount of the alpha ketoacid by-product or ketone by-product in a reduction step, wherein more than 90% of the PPO is converted to L-glufosinate.
2. The method of embodiment 1, wherein more than 91% of the PPO is converted to L-glufosinate.
3. The method of embodiment 1, wherein more than 92% of the PPO is converted to L-glufosinate.
4. The method of embodiment 1, wherein more than 93% of the PPO is converted to L-glufosinate.
5. The method of embodiment 1, wherein more than 94% of the PPO is converted to L-glufosinate.
6. The method of embodiment 1, wherein more than 95% of the PPO is converted to L-glufosinate.
7. The method of embodiment 1, wherein more than 96% of the PPO is converted to L-glufosinate.
8. The method of embodiment 1, wherein more than 97% of the PPO is converted to L-glufosinate.
9. The method of embodiment 1, wherein more than 98% of the PPO is converted to L-glufosinate.
10. The method of embodiment 1, wherein more than 99% of the PPO is converted to L-glufosinate.
11. The method of embodiment 1, wherein the DAAO enzyme is a mutant DAAO comprising one or more mutations at positions 54, 56, 58, 213, and 238, using SEQ ID NO:8 as a reference sequence, wherein the mutation at position 54 is selected from the group consisting of N54C, N54L, N54T, and N54V; the mutation at position 56 is T56M or T56N; and the mutation at position 58 is selected from the group consisting of F58A, F58G, F58H, F58K, F58N, F58Q, F58R, F58S, and F58T. 12. The method of embodiment 11, wherein the mutations comprise N54V, T56N, F58H, and M213S.
13. The method of embodiment 1, wherein the alpha ketoacid by-product is reduced by enzymatic conversion.
14. The method of any one of embodiments 1-13, wherein the TA enzyme is an enzyme encoded by SEQ ID NO:6 or SEQ ID NO:7.
15. The method of any one of embodiments 1-13, wherein the TA enzyme is a GabT transaminase having the amino acid sequence set forth in SEQ ID NO:6 or SEQ ID NO:7.
16. The method of any one of embodiments 1-15, wherein the amine donor is glutamate or L-glutamate.
17. The method of embodiment 16, wherein the alpha ketoacid by-product is reduced in amount by the addition of a ketoglutarate decarboxylase (KGD) enzyme.
18. The method of any one of embodiments 1-17, wherein the oxidation, the amination, and the reduction steps are performed in a single container.
19. The method of any one of embodiments 1-17, wherein all reagents are substantially added at the start of the reaction.
20. The method of embodiment 18, wherein the reagents for the oxidation, the amination, and the reduction steps are added to the single container at different times.
21. The method of any one of embodiments 1-17, wherein the oxidation, the amination, and the reduction steps are performed in separate containers.
22. The method of any one of embodiments 1-17, wherein one or more of the enzymes are immobilized.
23. The method of any one of embodiments 1-17, wherein the by-product is sequestered from the reaction by binding or further conversion to another product.
24. A method for converting PPO to L-glufosinate comprising aminating the PPO by a transaminase (TA) enzyme, using an amine group from one or more amine donors to form L-glufosinate and an alpha ketoacid or ketone by-product, and reducing the amount of the alpha ketoacid or the ketone by-product wherein more than 90% of the PPO is converted to L-glufosinate.
25. The method of embodiment 24, wherein the alpha ketoacid or the ketone by-product is reduced in amount by the addition of an enzyme which reacts with the by-product.
26. The method of embodiment 25, wherein the amine donor is L-glutamate.
27. The method of embodiment 25, wherein the enzyme which reacts with the by-product is KGD.
28. The method of embodiment 27, wherein the KGD comprises the one of the sequences set forth in SEQ ID NO:1 to 5.
29. The method of embodiment 24, wherein the TA enzyme is an enzyme encoded by SEQ ID NO:6 or SEQ ID NO:7.
30. The method of embodiment 24, wherein the TA enzyme is a GabT transaminase having the amino acid sequence set forth in SEQ ID NO:6 or SEQ ID NO:7.
31. The method of embodiment 24, wherein the TA enzyme has lower affinity for SSA than for PPO.
32. A composition comprising L-glufosinate and succinic semialdehyde (SSA).
33. A composition comprising L-glufosinate ammonium in an amount from 10-30% by weight of the composition; one or more additional components selected from the group consisting of sodium alkyl ether sulfate in an amount from 10-40% by weight of the composition; 1-methoxy-2-propanol in an amount from 0.5-2% by weight of the composition; dipropylene glycol in an amount from 4-18% by weight of the composition; and alkyl polysaccharide in an amount from 4-20% by weight of the composition.
34. A composition comprising L-glufosinate ammonium in an amount from 10-30% by weight of the composition; one or more additional components selected from the group consisting of sodium alkyl ether sulfate in an amount from 10-40% by weight of the composition; propylene glycol in an amount from 5-20% by weight of the composition; and alkyl polysaccharide in an amount from 4-20% by weight of the composition.
35. A method for selectively controlling weeds in an area comprising: applying an effective amount of a composition comprising L-glufosinate at an enantiomeric excess of greater than 99% over D-glufosinate to the area.
36. A composition comprising L-glufosinate, PPO, and SSA, wherein the PPO is present in an amount less than 10% of the amount of L-glufosinate and SSA is present at a molar amount up to 2 times the amount of L-glufosinate.
37. A composition comprising greater than 90% L-glufosinate, less than 10% PPO, less than 10% of the alpha ketoacid or ketone by-product, and at least 25% of the conversion product relative to the amount of L-glufosinate.

The following examples are offered by way of illustration and not by way of limitation.

### Examples

### Example 1: Activity of Selected KGD Enzymes Utilizing KG as a Substrate

In this example, various KGD enzymes are tested for the activity of degrading KG into SSA. The following reactants were added at the start of the 470 uL reaction: 15 mM KG, 107 uM thiamine pyrophosphate (TPP), 4.5 mM MgCl₂-6H₂O, 63 to 197 ug/mL KGD enzyme, 30 mM Kphos (pH 8). Reactions were incubated at 30° C. with 300 rpm shaking for 21 hours, and then left on the bench for 5 hours before HPLC analysis. The degradation of KG overtime is shown in Table 1 after normalization of starting KG concentrations.

TABLE 1

| Time (hours) | M. smegmatis | S. apiospermum | M. maritypicum | A. enclensis | K. phytohabitans |
|---|---|---|---|---|---|
| 0 | 15.0 | 15.0 | 15.0 | 15.0 | 15.0 |
| 2 | 11.9 | 13.8 | 6.8 | 8.7 | 9.5 |
| 4 | 9.4 | 13.3 | 1.7 | 4.3 | 5.3 |
| 26 | 0.7 | 3.9 | 1.0 | 1.2 | 1.0 |

### Example 2: Activity of Selected KGD Enzymes Utilizing PPO as a Substrate

In this example, various KGD enzymes are tested for the activity of decarboxylating PPO. The following reactants were added at the start of the 500 uL reaction: 40 mM PPO, 40 mM L-glufosinate, 0.1 mM thiamine pyrophosphate (TPP), 4.2 mM MgCl₂-6H₂O, ˜30-90 ug/mL KGD enzyme. Reactions were incubated at 30° C. with 300 rpm shaking for 4 hours. Reactions were then left on the bench for 71 hours, at which point an end point sample was taken. The normalized degradation of PPO over time is shown in Table 2.

TABLE 2

| Time (hours) | M. smegmatis | S. apiospermum | M. maritypicum | A. enclensis | K. phytohabitans |
|---|---|---|---|---|---|
| 0 | 40.0 | 40.0 | 40.0 | 40.0 | 40.0 |
| 2 | 40.5 | 39.4 | 45.0 | 42.2 | 40.1 |
| 4 | 40.3 | 36.0 | 40.8 | 35.0 | 39.8 |
| 75 | 42.4 | 34.8 | 38.3 | 35.4 | 38.5 |

### Example 3: Activity of Wild Type and Mutant TA Enzymes in the L-Glufosinate Reaction

In this example, wild type E. coli gabT TA enzyme and its E211S variant were tested for activity on the desired reaction of converting PPO and glutamate to KG and L-glufosinate. The following reactants were added at the start of the 500 uL reaction: 25 mM PPO, 25 mM L-glufosinate, 180 mM L-glutamate (3.6×molar excess of PPO+Glu-L), 0.2 mM PLP, 25 mM Kphos (pH 8.0) to bring to pH˜7.5, 75 μg/mL gabT. Reactions were incubated at 30° C. with 300 rpm shaking and samples taken for HPLC analysis. The normalized appearance of KG over time is shown in Table 3.

TABLE 3

| Time (hours) | Wild type | E211S mutant |
|---|---|---|
| 0.0 | 0.0 | 0.0 |
| 0.2 | 10.9 | 2.0 |
| 0.5 | 14.2 | 5.1 |
| 1.0 | 14.5 | 7.9 |
| 5.0 | 14.4 | 14.1 |
| 24.5 | 14.5 | 14.8 |

### Example 4: Activity of Wild Type and Mutant TA Enzymes in their Native Reactions

In this example, wild type E. coli gabT TA enzyme and its E211S variant were tested for activity on the native reaction of converting SSA and glutamate to KG and GABA. The following reactants were added at the start of the 500 uL reaction: 250 mM L-glutamate, 50 mM SSA, 0.2 mM PLP, 50 mM Kphos (pH 8.0) and 30 mM NaOH to bring to pH 7.5, 75 ug/mL gabT enzyme. Reactions were incubated at 30° C. with 300 rpm shaking and samples taken for HPLC analysis. The normalized appearance of KG over time is shown in Table 4.

TABLE 4

| Time | | |
|---|---|---|
| (minutes) | Wild type | E211S mutant |
| 0 | 0.0 | 0.0 |
| 10 | 12.4 | 0.0 |
| 30 | 13.2 | 0.0 |
| 60 | 13.5 | 0.5 |
| 1200 | 15.5 | 4.9 |

### Example 5: Improved Yield of L-Glufosinate with the Addition of KGD Enzyme

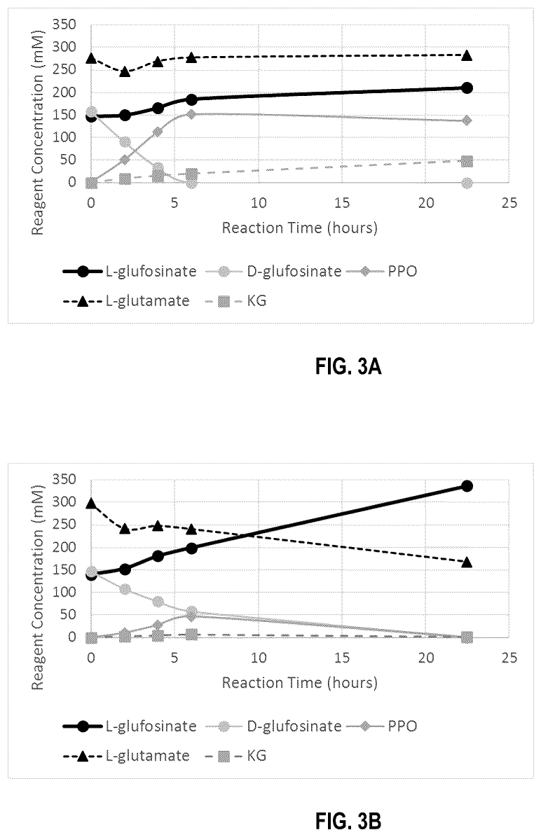

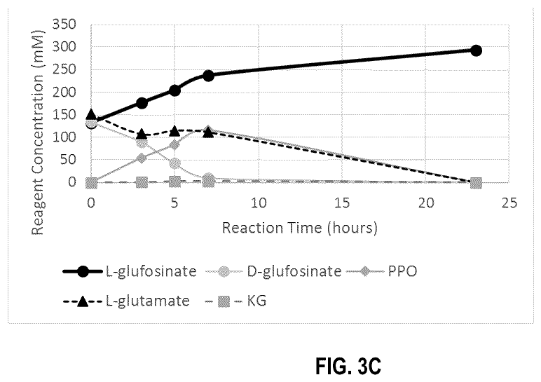

In this example, racemic glufosinate is deracemized using a DAAO enzyme, a TA enzyme, and with or without, a KGD enzyme. The following reactants were added at the start of the 500 uL reaction: 300 mM racemic glufosinate, 150 or 300 mM L-glutamate, 0.1 mM thiamine pyrophosphate (TPP), 4.2 mM MgCl₂-6H₂O, 0.2 mM PLP, 0.2 mg/mL catalase lysate, 1 mg/mL DAAO (pure enzyme), 0.4 mg/mL E211S gabT (2.35 mg/mL CFE), +/−0.5 mg/mL KGD from A. enclensis (7.15 mg/mL CFE), and ˜35 mM Kphos (pH 8) and 5 mM NaOH to adjust pH to ˜7.5. Reactions were incubated open at 30° C. with 320 rpm shaking for the first 6 to 7 hours. Tubes were closed for overnight incubation to minimize evaporation. FIG. 3 shows the progress of the reaction without (A) or with (B and C) the addition of the KGD enzyme and a starting L-glutamate concentration of 300 mM (A and B) or 150 mM (C). In the graphs are shown the concentrations of L-glufosinate (black circles, black bold line), D-glufosinate (light grey circles, light grey solid line), PPO (dark grey diamonds, dark grey solid line), L-glutamate (black triangles, black dashed line), and KG (dark grey squares, dark grey dashed line).

### Example 6: Improved Yield of L-Glufosinate with Mutant TA Enzyme

In this example, a mutant TA enzyme with reduced affinity for SSA as a substrate is used in a deracemization and the yield of L-glufosinate is higher than the wild type TA enzyme, even though the initial rate of production is higher for the wild type TA. The following reactants were added at the start of the 500 uL reaction: 300 mM D,L-Glufosinate, 150 mM L-Glutamate (MSG). 0.1 mM thiamine pyrophosphate (TPP), 4.2 mM MgCl₂-6H₂O, 0.2 mM pyridoxal phosphate (PLP), 0.2 mg/mL catalase powder (Sigma), 1 mg/mL DAAO Ac302, 0.4 mg/mL TA (SEQ ID NO:6 is wild type, SEQ ID NO:7 is E211S mutant), 0.36 mg/mL A. enclensis KGD (SEQ ID NO:4), ˜30 mM Kphos (pH 8), and 10 mM NaOH to adjust pH to 7.7. Reactions were incubated open at 30° C. with 320 rpm shaking for the first 4 hours. Tubes were closed for overnight incubation to minimize evaporation. The normalized amount of L-glufosinate (in millimolar) over time is shown in Table 5.

TABLE 5

| Time (hours) | Wild type | E211S mutant |
|---|---|---|
| 0 | 146 | 145 |
| 2 | 171 | 153 |
| 4 | 194 | 164 |
| 23 | 233 | 259 |

### Example 7: Improved Yield of L-Glufosinate with the Addition of Various KGD Enzymes

In this example, racemic glufosinate is deracemized using a DAAO enzyme, a TA enzyme, and with or without, various KGD enzymes. The following reactants were added at the start of the 500 uL reaction: 300 mM D,L-Glufosinate, 150 mM L-Glutamate (MSG). 0.1 mM thiamine pyrophosphate (TPP), 4.2 mM MgCl₂-6H₂O, 0.2 mM pyridoxal phosphate (PLP), 0.2 mg/mL catalase powder (Sigma), 1 mg/mL DAAO Ac302, 0.4 mg/mL TA (E211S mutant), 0.5 mg/mL KGD (0.36 mg/mL for A. enclensis KGD, 0 mg/mL for No KGD), ˜30 mM Kphos (pH 8), and 10 mM NaOH to adjust pH to 7.7. Reactions were incubated open at 30° C. with 320 rpm shaking for the first 4 hours. Tubes were closed for overnight incubation to minimize evaporation. The percent of PPO converted to L-glufosinate over time is shown in Table 6.

TABLE 6

| | | M. smegmatis | S. apiospermum | M. maritypicum | A. enclensis | K. phytohabitans |
|---|---|---|---|---|---|---|
| Time | No | SEQ ID | SEQ ID | SEQ ID | SEQ ID | SEQ ID |
| (hours) | KGD | NO: 1 | NO: 2 | NO: 3 | NO: 4 | NO: 5 |
| 0 | 0 | 0 | 0 | 0 | 0 | 0 |
| 2 | 14 | 16 | 49 | 41 | 17 | 45 |
| 4 | 12 | 31 | 63 | 48 | 22 | 48 |
| 23 | 22 | 85 | 96 | 97 | 87 | 94 |

### Example 8: Activity of KGD Enzymes Immobilized on HA403 Resin

In this example, KG is decarboxylated using various immobilized KGD enzymes. The following reactants were added at the start of the 2.5 mL reaction: 100 mM KG, 0.17 mM TPP, 4 mM MgCl₂-6H2O, 2 mg/mL KGD immobilized on 0.25 g ReliZyme HA403 resin (Resindion), 100 mM Kphos (pH 7), 177.5 mM NaOH to pH 6.47 at 23° C. Reactions were incubated in closed 50 mL tubes at 30° C. with 275 rpm shaking. The concentration of KG over time is shown in Table 7.

TABLE 7

| | S. | M. | A. | K. |
|---|---|---|---|---|
| | apiospermum | maritypicum | enclensis | phytohabitans |
| Time | SEQ ID | SEQ ID | SEQ ID | SEQ ID |
| (hours) | NO: 2 | NO: 3 | NO: 4 | NO: 5 |
| 0 | 95 | 98 | 90 | 94 |
| 4 | 79 | 45 | 57 | 48 |
| 21 | 34 | 0 | 7 | 0 |
| 27 | 34 | 0 | 3 | 0 |
| 43.5 | 29 | 0 | 0 | 0 |

### Example 9: Activity of KG a Enzymes Immobilized on HA403 Resin

In this example, KG is decarboxylated using various immobilized KGD enzymes. The following reactants were added at the start of the 2.5 mL reaction: 100 mM KG, 0.1 mM TPP, 4 mM MgCl₂-6H₂O, 1 mg/mL KGD immobilized on 0.5 g Lifetech ECR8209F (Purolite), 100 mM Kphos (pH 7), 170 mM NaOH to pH 6.5 at 2° C. Reactions were incubated in closed 50 mL tubes at 30° C. with 275 rpm shaking. The concentration of KG over time is shown in Table 8.

TABLE 8

| Time | M. smegmatis | S. apiospermum | M. maritypicum | A. enclensis | K. phytohabitans |
|---|---|---|---|---|---|
| (hours) | SEQ ID NO: 1 | SEQ ID NO: 2 | SEQ ID NO: 3 | SEQ ID NO: 4 | SEQ ID NO: 5 |
| 0 | 96 | 95 | 96 | 97 | 98 |
| 3 | 80 | 61 | 74 | 94 | 64 |
| 6 | 62 | 40 | 53 | 89 | 40 |
| 70 | 20 | 0 | 1 | 34 | 0 |

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

What is claimed is:

1. A method for converting PPO to L-glufosinate, comprising:
reacting D-glufosinate with a D-amino acid oxidase (DAAO) enzyme in the presence of oxygen to form PPO (2-oxo-4-(hydroxy(methyl)phosphinoyl)butyric acid) in an oxidation step; and
aminating the PPO by a transaminase (TA) enzyme, using isopropylamine to form L-glufosinate and acetone by-product;
wherein more than 90% of the PPO is converted to L-glufosinate,
wherein the DAAO enzyme is a mutant DAAO comprising mutations at positions 54, 56, 58, and 213 using SEQ ID NO:8 as a reference sequence,
wherein the mutations comprise N54V, T56N, F58H, and M213S, and
wherein the TA enzyme is an enzyme encoded by SEQ ID NO:7.

2. The method of claim 1, wherein the amount of the acetone by-product is reduced by addition of an enzyme which reacts with the by-product.

3. A method for making L-glufosinate, comprising:
reacting D-glufosinate with a D-amino acid oxidase (DAAO) enzyme to form PPO (2-oxo-4-(hydroxy(methyl)phosphinoyl)butyric acid) while aerating in an oxidation step; and
aminating the PPO by a transaminase (TA) enzyme, using isopropylamine to form L-glufosinate and an acetone by-product in an amination step;
wherein more than 90% of the PPO is converted to L-glufosinate,
wherein the DAAO enzyme is a mutant DAAO comprising mutations at positions 54, 56, 58, and 213 using SEQ ID NO:8 as a reference sequence,
wherein the mutations comprise N54V, T56N, F58H, and M213S, and
wherein the TA enzyme is an enzyme encoded by SEQ ID NO:7.

4. The method of claim 3, wherein more than 98% of the PPO is converted to L-glufosinate.

5. The method of claim 3, wherein the oxidation, the amination, and the reduction steps are performed in a single container.

6. The method of claim 3, wherein all reagents are substantially added at the start of the reaction.

7. The method of claim 5, wherein reagents for the oxidation, the amination, and the reduction steps are added to the single container at different times.

8. The method of claim 3, wherein the oxidation, the amination, and the reduction steps are performed in separate containers.

9. A composition comprising L-glufosinate and less than 10% acetone by-product relative to the amount of L-glufosinate.

10. The composition of claim 9, comprising L-glufosinate, less than 10% PPO and less than 10% acetone by-product, relative to the amount of L-glufosinate.

11. The composition of claim 9, comprising greater than 90% L-glufosinate, less than 10% PPO, less than 10% acetone by-product, relative to the amount of L-glufosinate.
